(19) United States
(12) Patent Application Publication
(10) Pub. No.: US 2010/0130590 A1
(43) Pub. Date: May 27, 2010

(54) DELETION BEARING BARD1 ISOFORMS AND USE THEREOF

(75) Inventors: Irmgard Irminger-Finger, Geneva (CH); Stephan Ryser, Geneve (CH); Lin Li, Geneve (CH)

Correspondence Address: SALIWANCHIK LLOYD & SALIWANCHIK;A PROFESSIONAL ASSOCIATION, PO Box 142950, GAINESVILLE, FL, 32614, US

(73) Assignee: Hopitaux Universitaires De Geneve, Geneva 4 (CH)

(21) Appl. No.: 12/593,394

(22) PCT Filed: Apr. 1, 2008
(86) PCT No.: PCT/EP08/53881

§ 371 Date: Sep. 28, 2009

Related U.S. Application Data
(60) Provisional application No. 60/907,432, filed on Apr. 2, 2007.

(51) Int. Cl. A61K 31/7088; C12Q 1/02; G01N 33/53; C07K 14/435; C07H 21/04; C12P 21/06; C07K 16/00; C12N 15/63

(52) U.S. Cl. 514/44 A; 435/29; 435/7.1; 530/350; 536/23.5; 435/69.1; 536/24.31; 536/24.33; 530/387.9; 435/320.1

(57) ABSTRACT

The present invention relates to new protein isoforms, use thereof, methods of preparation thereof, methods of detection thereof, antibodies thereof, combination of antibodies thereof, use of these antibodies and combinations thereof and use of antagonists of those isoforms for the treatment of gynaecological cancers.

### FIELD OF THE INVENTION

The present invention relates to new protein isoforms, use thereof, methods of preparation thereof, methods of detection thereof, antibodies thereof, combination of antibodies thereof, use of these antibodies and combination thereof and use of antagonists of those isoforms for the treatment of gynaecological cancers.

### BACKGROUND OF THE INVENTION

The tumor suppressor BARD1 (BRCA1 Associated Ring Domain) has multiple functions with and without BRCA1. N-terminal RING finger domains of BARD1 and BRCA1 confer an interaction module, and are essential for heterodimer formation. Mutations disrupting this interaction are found to be associated with cancer, indicating that the heterodimer has essential tumor suppressor functions, presumably attributable to its ubiquitin ligase activity. By itself, BARD1 has a function in apoptosis by stabilizing p53 and facilitating its phosphorylation, another important tumor suppressor function. BARD1 and BRCA1 were also reported to be involved in mitosis and specifically spindle formation.

Mutations and upregulated expression of BARD1 were found in breast and ovarian cancer. They were associated with poor prognosis, suggesting that cancer-associated BARD1 might be deficient in tumor suppressor functions. RT-PCR was performed to characterize the cDNA structure of cancer-associated BARD1 iso forms in breast, ovarian, cervical, and uterine cancers and cancer cell lines. Interestingly, all gynaecological cancers expressed a number of BARD1 isoforms derived from differential splicing, which was not the case for non-gynaecological cancers such as lymphomas. In cervical cancers, however, differentially spliced isoforms were not found but a truncated transcript, derived from alternative transcription initiation. Ovarian cancer and uterine cancer cells expressed a mixture of iso forms generated by both mechanisms. Specific repression of isoforms in a cancer cell line deficient of full length BARD1 leads to a complete growth arrest. This suggests that isoforms, which are expressed in gynaecological cancers and lack the central part and exons encoding the BRCA1-interacting RING finger domain, are essential for tumor cell growth.

BARD1 was found to be an interacting protein with BRCA1 (Wu et al., 1996, Nat. Genet. 14, 430-440). BARD1 and BRCA1 form a stable heterodimer with function in DNA repair, transcription regulation, RNA processing, ubiquitination and cell cycle regulation (Irminger-Finger et al., 2006, Nature Reviews 6, 382-391). Depletion of BARD1 leads to genomic instability, loss of polarity, premalignant phenotype, and embryonic lethality in knock out mice. As a tumour suppressor, BARD1 (SEQ ID NO: 1) also has a BRCA1 independent function in mediating p53-dependent apoptosis (Irminger-Finger et al., 2001, Molecular cell 8, 1255-1266). It binds to p53, facilitating its phosphorylation and stabilisation (Feki et al., 2005, Oncogene 24, 3726-3736). Recently a novel function of BARD1 in mitosis was found. The role of BRCA1/BARD1 in mitotic spindle assembly may contribute to its function in maintaining chromosome stability and tumour suppression. Furthermore, BARD1, by interacting with acidic coiled-coil protein TACC1, BRCA1, BRCA2 and Aurora B, plays a role in controlling mitosis completion and genetic stability.

BARD1 is expressed in most proliferative tissues, with maximum expression in spleen and testis (Ayi et al., 1998, Oncogene 17, 2143-2148). Furthermore, it is upregulated in response to hypoxia, and genotoxic stress (Irminger-Finger et al., 2001, Molecular cell 8, 1255-1266; Jefford et al., 2004, Oncogene 23, 3509-3520), and hormone signalling (Feki et al., 2005, above). This upregulation of BARD1 thus induces apoptosis pathways and tumour suppression (Irminger-Finger et al., 2001, above).

More than 600 mutations, comprising deletions, insertions miss-sense, and nonsense mutation have been identified in BRCA1. Since BARD1 is a tumour suppressor both as a heterodimer with BRCA1 and on its own, BARD1 mutations should also predispose to cancer. However, BARD1 mutations are less frequent. After screening a panel of sporadic breast, ovarian and endometrial cancers, three missense alterations were identified in the BARD1 gene at the amino acid positions Q564H, V695L, and S761N (That et al., 1998, Human Molecular Genetics 7, 195-202). Five alterations were discovered in an Italian cohort with familial breast and ovarian cancers that was chosen for its absence of BRCA1 and BRCA2 gene alterations in its proband (Ghimenti et al., 2002, Genes, chromosomes & cancer 33, 235-242). Apart from mutation, BARD1 shows aberrantly elevated expression and localization to the cytoplasm in cancer cells, as compared to the normal tissue where it is localized to the nucleus. Elevated BARD1 staining in the cytoplasm was correlated with poor prognostic factor for breast and ovarian cancer (Wu et al., 2006, Int. J. Cancer 118, 1215-1226).

Consistent with BARD1 isoform lacking exon 2 through 6 as well as no full length (FL), BARD1 was found in a rat ovarian cancer cell line that is resistant to apoptosis (Feki et al., 2005, above). This isoform lacks most of the RING domain and the entire ankyrin repeats, a region required for the apoptosis and p53 binding (Feki et al., 2005, above). The same iso form was later reported in Hela cells. Deletion of N-terminal epitopes of BARD1 was also found in majority of ovarian cancer (Wu et al., 2006, above). It was therefore hypothesized that specific isoforms of BARD1 might have lost its tumour suppressor functions and acquired tumourigenic properties. To elucidate BARD1 function in cancer, experiments were performed to characterize BARD1 expression pattern in various types of cancer and determine their structure and potential function in cancer cell growth (Li et al., 2007, Int. J. Biochem. Cell. Biol. 39(9):1659-1672).

Diagnostics and therapies of gynaecological diseases comprise some of the most severe unmet clinical needs, including breast, ovarian, cervical and uterine cancers. Therefore, there is a need for developing new substances and related methods for better diagnosing and treating such diseases.

### SUMMARY OF THE INVENTION

The present invention is directed towards to new protein isoforms, antibodies thereof, and related methods useful for the treatment of gynaecological cancers.

It is an object of the invention to provide new protein isoforms, antibodies thereof and related methods which are suitable for or the treatment of and/or prevention of and/or delaying the progression of gynaecological cancers, notably breast, ovarian, cervical and uterine cancers.

A first aspect of the invention provides a method for detecting the presence of gynaecological cancer related proteins (including breast cancer, ovarian cancer, endometrial and cervical cancer) according to any one of claims 1 to 27.

A second aspect of the invention provides an isolated polypeptide according to any one of claims 28 to 32.

A third aspect of the invention provides an isolated nucleic acid consisting of a nucleotide sequence according to any one for claims 33 to 34, recombinant expression vectors thereof, host cells transfected or transformed with a recombinant expression vector according to the invention and a process for producing cells capable of expressing a polypeptide according to the invention.

A fourth aspect of the invention provides the use of a nucleic acid according to the invention.

A fifth aspect of the invention provides an isolated antibody according to any one of claims 37 to 38.

A sixth aspect of the invention resides in a combination of antibodies according to any one of claims 39 to 46 and use thereof.

A seventh aspect of the invention provides a method for detecting the level of cellular expression of proteins according to claim 47.

An eighth aspect of the invention resides in the use of an antibody or a combination of antibodies according to the invention in an assay.

A ninth aspect of the invention provides a recombinant vector comprising a nucleic acid according to the invention.

A tenth aspect of the invention resides in a host cell transfected with the recombinant vector according to the invention.

An eleventh aspect of the invention provides a process for producing cells capable of expressing a polypeptide according to the invention.

A twelfth aspect of the invention resides in a kit comprising at least one polypeptide according to the invention. In a preferred embodiment, the kit according to the invention is useful for the detection of at least one gynaecological cancer related protein in a biological sample of a subject suspected of or suffering from a gynaecological cancer or at high risk of developing a gynaecological cancer.

A thirteenth aspect of the invention provides an immunoassay kit for detecting gynaecological cancer in a biological sample, the kit comprising at least one antibody according to the invention or a fragment thereof or a combination of antibodies according to the invention. In a preferred embodiment, the immunoassay kit according to the invention is useful for detection of at least one gynaecological cancer related protein in a biological sample of a subject suspected of or suffering from a gynaecological cancer.

A fourteenth aspect of the invention resides in the use of an antagonist of a polypeptide according to the invention for the manufacture of a medication for the treatment of a gynaecological cancer, including breast, ovarian, cervical and uterine cancers. In a preferred embodiment, the antagonist is an antibody according to the invention.

A fifteenth aspect according to the invention provides a method of treating a disease comprising the administration of a therapeutically effective amount of an antagonist of a polypeptide according to the invention in a mammal in need thereof; wherein the disease is a gynaecological cancer, including breast, ovarian, cervical and uterine cancers.

Other features and advantages of the invention will be apparent from the detailed description, figures and sequence listings.

### DESCRIPTION OF THE FIGURES

FIG. 1. Structure of BARD1 isoforms. (A) RTPCR amplification of FL BARD1 coding region in normal skin fibroblast and Hela cells. (B) Diagram of BARD1 exons and structural domains compared to exon structure of FL BARD1 and isoforms α, β, γ, φ, δ, ε, and η. Approximate locations of structural domains are indicated as RING, Ankyring, and BRCT above BARD1 molecule structure. Small arrows mark positions of forward and reverse primers used for RT-PCR. Open reading frame corresponding to known BARD1 sequence is presented by empty boxes, alternative reading frame is indicated as spotted boxes. Amino acids and calculated molecular weight are indicated. The respective sequence IDs are listed on the left side for DNA sequences and on the right side for protein sequences (C) Sequences of splice junctions of isoforms β, γ, and η are presented. Known BARD1 ORF is marked with a grey bar, alternative ORF with an empty bar. Possible translation initiation methionines are labelled black bar (underlined) within alternative ORF of isoforms β, γ, and η. The sequence IDs are indicated.

FIG. 2. RT-PCR of breast cancer cell lines (B1-B9) for amplification of FL BARD1. Hela cells were used as a control.

FIG. 3. RT-PCR of cervical cancer cell lines (C1-C9) for amplification of regions as indicated. Nucleotide position of the forward primers are indicated. Hela cells were used as a control.

FIG. 4. Amplification of FL BARD1 and truncated isoform from exon 4 through exon 11 in endometrial and ovarian cancer cell lines. (A) RT-PCR in endometrial cancer cell lines. (B) RT-PCR in ovarian cancer cell lines. Hela cells were used as a control.

FIG. 5. RT-PCR of BARD1 expression in haematology tumour cell lines (H1-H13). No splice isoforms are visible.

FIG. 6. Alternative initiation of transcription in exon 4. (A) Nested PCR with 5′ GeneRacer of ovarian cancer sample and Hela cells. Forward primer was 5′ nested primer and reverse primer located in exon 6. The bands sequenced were indicated by arrows. (B) mRNA and protein sequence of BARD1 exon 4. Positions of new initiations of transcription found by 5′ GeneRacer are indicated (Start 1, 2 and 3). (C) Diagram of BARD1 structure and three new transcripts initiation isoforms (Ω1, Ω2, Ω3). Primers and antibodies used in the experiment were shown. The translated regions were shown in thick lines, non-translated in thin lines.

FIG. 7. Western blot of ovarian cancer cell lines probed with BARD1 antibodies H300 and JH3 in ovarian cancer cell lines. MW of different BARD1 isoforms was indicated. Hela cells were used as a control.

FIG. 8. Immunohistochemical staining of ovarian cancer tissue arrays. (A) Correlation of BARD1 expression and tumour size in ovarian cancer. (B) Correlation of BARD1 expression and lymph node metastasis in ovarian cancer. (C) Immunohistochemistry of a patient in stage T3 showed both N19 and WFS were negative while C20 was strongly positive, which indicates that only omega iso forms are expressed. (D) Correlation of BARD1 expression with different pathology grades in ovarian cancer.

FIG. 9. BARD1 expression in different pathologic types of ovarian cancer. (A) Immunohistochemical staining in different pathologic types. Clear cell carcinoma has the highest score. SeC, serous carcinoma; EnC, endometriod carcinoma; CCC, clear cell carcinoma; MuC, mucinous carcinoma. (B) RT-PCR for amplification of FL BARD1 in clear cell carcinoma cell line. (C) Immunohistochemistry of clear cell carcinoma showed strong staining by both N19 and C20, but was negative for WFS.

FIG. 10. Function of isoforms in cell viability. (A) Western Blot probed with BARD1 antibody H300 showed only iso forms in NuTu cells (rat ovarian cancer). (B) RT-PCR showed that BARD1 expression was repressed by siRNA78. (C) Fluorescence microscopy of GFP and DAPI in NuTu cells transduced with siRNAs-GFP constructs. (D) Histogram of survival cells in si78 (targeting exon 9, repressing isoform) and si34 (targeting exon 2) transduced NuTu cells.

FIG. 11. RT-PCR of BARD1 expression in lung cancer cell lines. Hela cells were used as a control. No splice isoforms are visible.

### DETAILED DESCRIPTION OF THE INVENTION

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or relieving the disease, i.e., causing regression of the disease and/or its symptoms or conditions.

The term “subject” as used herein refers to mammals. For examples, mammals contemplated by the present invention include human, primates, domesticated animals such as cattle, sheep, pigs, horses and the like.

The term “isolated” is used to indicate that the molecule is free of association with other proteins or polypeptides, for example as a purification product of recombinant host cell culture or as a purified extract.

The term “antibody” comprises antibodies binding to at least one isoform according to the invention or fragment thereof, chimeric antibodies recognizing and/or binding selectively to at least one iso form according to the invention or fragment thereof, fully human, humanized, genetically engineered or bispecific or multispecific antibodies as well as fragments thereof such as single chain antibodies (scFv) or domain antibodies against at least one isoform according to the invention or fragment thereof and the like. Antibodies of this invention may be monoclonal or polyclonal antibodies, or fragments or derivative thereof having substantially the same antigen specificity. The term “selectively” indicates that the antibodies preferentially recognize and/or bind to at least one target polypeptide or epitope of an isoform according to the invention, i.e., with a higher affinity than any binding to any other antigen or epitope, i.e. the binding to the target polypeptide can be discriminated from non-specific binding to other antigens such as other proteins not belonging to the group of the iso forms according to the invention. Examples of antibodies or combinations thereof according to the invention are presented herein. The binding affinity of an antibody can be readily determined by one of ordinary skill in the art, for example, by Scatchard analysis (Scatchard et al., 1949, Ann NY Acad. Sci., 51, 660-672).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

The term “antagonists” is defined as a molecule that antagonizes completely or partially one or more activity of biological molecule. Preferred antagonists according to the invention antagonize the biological function of at least of the iso forms according to the invention and does not antagonize FL BARD1 biological activity. The term “antagonist” includes but is not limited to: BARD 1 iso forms specific antibodies of any sort (polyclonal, monoclonal, antibody fragments, antibody variants), chimaeric proteins, natural or unnatural proteins with BARD 1 iso form antagonizing activities, small molecules, nucleic acid derived polymers (such as DNA and RNA aptamers, siRNAs, PNAs, or LNAs), peptidomimetics, fusion proteins, or gene therapy vectors driving the expression of such antagonists. An antagonist, as an isolated, purified or homogeneous protein according to the invention, may be produced by recombinant expression systems as described herein or purified from naturally occurring cells.

Suitable expression of polypeptides according to the invention, variants or fragments, antagonists, thereof include prokaryotes, yeast or higher eukaryotic cells. Appropriate cloning and expression vectors for use with bacterial, fungal, yeast and mammalian cellular hosts are described for example in Pouwels et al., 1985, Cloning Vectors: A laboratory manual, Elsevier, N.Y.

The expression “gynaecological cancer” includes breast cancer, ovarian cancer, endometrial cancer and cervical cancer.

The expression “risk of developing a future gynecological cancer” refers to a higher risk of developing a gynaecological cancer than an individual (such as a mammal), who does not present the iso form.

The expression “biological sample” refers to cells, tissue samples or cell components (such as cellular membranes or cellular components) obtained from a subject suspected of or suffering from gynaecological cancer or at high risk of developing a gynaecological cancer. Examples include blood, serum, plasma and tissue samples.

The expression “kit” comprises at least one polypeptide according to the invention or at least one antibody according to the invention or a fragment thereof or a combination thereof as described herein coupled to a solid matrix and instructional material. The solid matrix as referred herein may include nitrocellulose paper, glass slide, microtitre plates and wells.

Table 1 below presents the Sequence identity numbers and associated molecules:

TABLE 1

| SEQ ID NO. | Molecule |
|---|---|
| 1 | Amino acid sequence for human BARD1 full length |
| 2 | Amino acid sequence for human BARD1- alpha |
| 3 | Amino acid sequence for human BARD1- beta (Reading frame 1 in exon 1) |
| 4 | Amino acid sequence for human BARD1- beta (Reading frame 2 in exon 1) |
| 5 | Amino acid sequence for human BARD1- gamma |
| 6 | Amino acid sequence for human BARD1- phi |
| 7 | Amino acid sequence for human BARD1- epsilon |
| 8 | Amino acid sequence for human BARD1- eta (Reading frame 1 in exon 1) |
| 9 | Amino acid sequence for human BARD1- eta (Reading frame 2 in exon 1) |
| 10 | Amino acid sequence for human BARD1- omega 1 |
| 11 | Amino acid sequence for human BARD1- omega 2 |
| 12 | Nucleotide sequence for human BARD1 full length |
| 13 | Nucleotide sequence for human BARD1 alpha (Exon 2 deleted), |
| | Exon 1 linked to exon 3; Exons (1-3-4-5-6-7-8-9-10-11); Exon 3 |
| | starts at 232 (TAATTGTGT . . . ), 2473 nucleotides, ATG at position |
| | 74, Translates into 758 amino acids starting “MPDNRQPRNR”. |
| | Calculated molecular weight 84.56 kDa |
| 14 | Nucleotide sequence for human BARD1 beta (Exons 2 and 3 |
| | deleted), Exon 1 linked to exon 4; Exons (1-4-5-6-7-8-9-10-11); |
| | Exon 4 starts at 232 (ATTTGAAAG . . . ), 2324 nucleotides, translates |
| | into 680 amino acids starting with “MVAVPGPTVA”. Calculated |
| | molecular weight: 75.46 kDa |
| 15 | Nucleotide sequence for human BARD1 gamma (Exon 4 deleted), |
| | Exon 3 linked to exon 5; Exons (1-2-3-5-6-7-8-9-10-11); Exon 5 |
| | starts at 438 (GGCGACATACC . . . ), 1456 nucleotides, translates |
| | into 126 amino acids. Calculated molecular weight: 14.34 kDa |
| 16 | Nucleotide sequence for human BARD1 phi (Exon 3-6 deleted), |
| | Exon 2 linked to exon 7; Exons (1-2-7-8-9-10-11); Exon 7 starts at |
| | 244 (TAATATATTTGG . . . ), 1008 nucleotides, translates into 327 |
| | amino acids starting with “MPDNRQPRNR”, Calculated molecular |
| | weight 37.13 kDa |
| 17 | Nucleotide sequence for human BARD1 epsilon (Exons 4-9 |
| | deleted), Exon 3 linked to exon 10; Exons (1-2-3-10-11); Exon 10 |
| | starts at 393 (GGGTAAAAGC . . . ), 825 nucleotides, translates into |
| | 263 amino acids. Calculated molecular weight: 30.36 kDa, starting |
| | with “MPDNRQPRNR” |
| 18 | Nucleotide sequence for human BARD1 eta (Exons 2-9 deleted), |
| | Exon 1 linked to Exon 10 (Exons1-10-11); Exon 10 starts at 232 |
| | (GGGTAAAA . . . ), 702 nucleotides, translates into 219 amino acids |
| 19 | Nucleotide sequence for human BARD1 omega 1, translates into |
| | 264 amino acids |
| 20 | Nucleotide sequence for human BARD1 omega 2, translates into |
| | 449 amino acids |
| 21 | Amino acid sequence for human BARD1 beta fragment |
| 22 | Amino acid sequence for human BARD1 gamma fragment |
| 23 | Nucleotide sequence for human BARD1 omega 3, translates into |
| | 347 amino acids |
| 24 | Amino acid sequence for human BARD1 omega 3 |
| 25 | Amino acid sequence for synthetic peptide 1 |
| 26 | Nucleotide sequence for 5′ primer from exon 11 |
| 27 | Nucleotide sequence for reverse primer from exon 11 |
| 28 | Nucleotide sequence for 5′ primer 1 from exon 6 |
| 29 | Nucleotide sequence for reverse primer from exon 6 |
| 30 | Amino acid sequence for synthetic peptide 2 |
| 31 | Nucleotide sequence for 5′ primer from exon 1 |
| 32 | Nucleotide sequence for reverse primer from exon 11 |
| 33 | Nucleotide sequence for 5′ primer from exon 3 |
| 34 | Nucleotide sequence for 5′ primer 1 from exon 4 |
| 35 | Nucleotide sequence for 5′ primer 2 from exon 4 |
| 36 | Nucleotide sequence for 5′ primer 3 from exon 4 |
| 37 | Nucleotide sequence for 5′ primer 4 from exon 4 |
| 38 | Nucleotide sequence for 5′ primer 2 from exon 6 |

According to one aspect of the invention, is provided a method for detecting the presence of gynaecological cancer related proteins (including breast cancer, ovarian cancer, endometrial and cervical cancer) in a biological sample, comprising the steps of:

(a) Determining one or more of the following in a sample from a female mammal (including tissue biopsies or blood samples):
i. The expression level of a protein of SEQ ID NO: 1 through a detectable signal proportional to the said level of expression; and
iia. The expression level of at least one protein of an amino acid sequence selected from the group consisting of SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9 through a detectable signal proportional to the said level of expression; and/or
iib. The expression and/or expression level of at least one protein of an amino acid sequence selected from the group consisting of SEQ ID NO: 10; SEQ ID NO: 11 and SEQ ID NO: 24 through a detectable signal proportional to the said level of expression;
   (b) Optionally comparing the expression levels obtained under step (i) with the expression level obtained under steps (iia) and/or (iib);
   (c) Detecting a signal indicative of a ratio lower than a 1:1 ratio between the expression level obtained under step (i) and the expression level obtained under steps (iia) and/or (iib); or detecting a signal indicative of the expression/expression level determined under step (iib).

According to a further aspect of the invention, is provided a method according to the invention comprising the steps of:

(a) Determining one or more of the following in a sample from a female mammal (including tissue biopsies or blood samples):
i. The expression level of a protein of SEQ ID NO: 1 through a detectable signal proportional to the said level of expression; and
iia. The expression level of at least one protein of an amino acid sequence selected from the group consisting of SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9 through a detectable signal proportional to the said level of expression; and/or
   (b) Optionally comparing the expression levels obtained under step (i) with the expression level obtained under steps (iia);
   (c) Detecting a signal indicative of a ratio lower than a 1:1 ratio between the expression level obtained under step (i) and the expression level obtained under step (iia).

According to another further aspect of the invention, is provided a method according to the invention, wherein the signal obtained under detection step (c) is indicative of a ratio lower than a 1:1 ratio between the expression level obtained under step (i) and the expression level obtained under step (iia).

According to another further aspect of the invention, is provided a method according to the invention, wherein the signal indicative of a ratio lower than a 1:1 ratio between the expression level obtained under step (i) and the expression level obtained under step (iia), obtained under step (c), is of or lower than a ratio about 1:2.

According to another further aspect of the invention, is provided a method according to the invention, wherein the signal obtained under the detection step (c) is indicative of a gynaecological cancer.

According to another further aspect of the invention, is provided a method according to the invention comprising the steps of:

(a) Determining one or more of the following in a sample from a female mammal (including tissue biopsies or blood samples):
i. The expression level of a protein of SEQ ID NO: 1 through a detectable signal proportional to the said level of expression; and
iib. The expression and/or expression level of at least one protein of an amino acid sequence selected from the group consisting of SEQ ID NO: 10; SEQ ID NO: 11 and SEQ ID NO: 24 through a detectable signal proportional to the said level of expression;
   (b) Optionally comparing the expression levels obtained under step (i) with the expression level obtained under step (iib);
   (c) Detecting a signal indicative of the expression/expression level obtained under step (iib).

According to another further aspect of the invention, is provided a method according to the invention, wherein the signal obtained under detection step (c) is indicative of a ratio lower than a 1:1 ratio between the expression level obtained under step (i) and the signal obtained under step (iib).

According to another further aspect of the invention, is provided a method according to the invention, wherein the signal indicative of a ratio lower than a 1:1 ratio between the expression level obtained under step (i) and the expression level obtained under steps (iib) is of or lower than a ratio about 1:5.

According to another further aspect of the invention, is provided a method according to the invention, wherein the signal obtained under the detection step (c) is indicative of a gynaecological cancer or a risk of developing a future gynaecological cancer in the subject.

According to another further aspect of the invention, is provided a method according to the invention, wherein the method further comprises a comparison step (d) of the expression levels obtained under steps (i), (iia) and/or (iib), respectively, with expression levels in a normal control, wherein the normal control includes expression levels measured in a biological sample from an individual not suspected to suffer from a gynaecological cancer.

According to another aspect of the invention, is provided a method for detecting the presence of gynaecological cancer related proteins (including breast cancer, ovarian cancer, endometrial and cervical cancer) in a biological sample, comprising the steps of:

(i) Reacting a sample from a female mammal (including tissue biopsy, blood sample) with at least one antibody, a fragment thereof or a combination thereof, which is specific to a protein of SEQ ID NO: 1; and
(ii) Reacting the said sample with at least one antibody, fragment thereof, or a combination thereof, which is specific to at least one protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9; and/or
(iii) Reacting the said sample with at least one antibody, a fragment thereof or a combination thereof, which is specific to at least one protein comprising an amino acid sequence selected from SEQ ID NO: 10, SEQ ID NO: 11 and SEQ ID NO: 24;
(iv) Detecting (a) a protein of SEQ ID: 1; and (b) a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9; and/or (c) a protein comprising an amino acid sequence selected from SEQ ID NO: 10, SEQ ID NO: 11 and SEQ ID NO: 24; wherein the detection is achieved through the detection of the interaction of each said antibody, fragment thereof or combination thereof, used under steps (i) and (ii) and/or (iii) with the corresponding said at least one protein, wherein the presence of the interaction correlates with the concentration of the protein in the biological sample;
(v) Detecting a signal indicative of a ratio lower than a 1:1 ratio between the said interaction detection signal obtained under step (iv) for a protein of SEQ ID NO: 1 and the said interaction detection signal obtained under step (iv) for either a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9; or for a protein comprising an amino acid sequence selected from SEQ ID NO: 10, SEQ ID NO: 11 and SEQ ID NO: 24; or detecting a signal indicative of an interaction signal detected under step (iv) for a protein comprising an amino acid sequence selected from SEQ ID NO: 10, SEQ ID NO: 11 and SEQ ID NO: 24.

According to a further aspect of the invention, is provided a method for detecting the presence of gynaecological cancer according to the invention, wherein the signal detected under step (v) indicative of a ratio lower than a 1:1 ratio between the said interaction detection signal obtained under step (iv) for a protein of SEQ ID NO: 1 and the said interaction detection signal obtained under step (iv) for a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9, is indicative of a gynaecological cancer.

According to another further aspect of the invention, is provided a method for detecting the presence of gynaecological cancer according to the invention, wherein the signal detected under step (v) indicative for a protein comprising an amino acid sequence selected from SEQ ID NO: 10, SEQ ID NO: 11 and SEQ ID NO: 24, is indicative of a gynaecological cancer or a risk of developing a future gynaecological cancer in the subject.

According to another further aspect of the invention, is provided a method for detecting the presence of gynaecological cancer according to the invention, wherein steps (ii) and/or (iii) further comprise a washing step (iiia) wherein the unbound antibodies are washed off from the sample.

According to another further aspect of the invention, is provided a method for detecting the presence of gynaecological cancer according to the invention, wherein the antibodies are conjugated to a detectable moiety.

According to another further aspect of the invention, is provided a method for detecting the presence of gynaecological cancer according to the invention, wherein the antibodies used under step (ii) is a combination of antibodies wherein the combination comprises (a) at least one antibody against exon 1 from full length BARD 1 (SEQ ID NO: 12); (b) at least one antibody against one exon selected from exon 4, exon 5, exon 6, exon 7, exon 8 and exon 9 from full length BARD 1 (SEQ ID NO: 12); and (c) at least one antibody against exon 10 and/or exon 11 from full length BARD 1 (SEQ ID NO: 12).

According to another further aspect of the invention, is provided a method for detecting the presence of gynaecological cancer according to the invention, wherein the antibodies used under step (ii) is a combination of antibodies wherein the combination comprises (a) at least one antibody against exon 1 from full length BARD 1 (SEQ ID NO: 12); (b) at least one antibody against one exon selected from exon 4, exon 5 and exon 6 from full length BARD 1 (SEQ ID NO: 12); and (c) at least one antibody against exon 10 and/or exon 11 from full length BARD 1 (SEQ ID NO: 12).

According to another further aspect of the invention, is provided a method for detecting the presence of gynaecological cancer according to the invention, wherein the antibodies used under step (ii) is a combination of antibodies wherein the combination comprises (a) at least one antibody against exon 1 from full length BARD 1 (SEQ ID NO: 12); (b) at least one antibody against exon 4 from full length BARD 1 (SEQ ID NO: 12); and (c) at least one antibody against exon 10 and/or exon 11 from full length BARD 1 (SEQ ID NO: 1).

According to another further aspect of the invention, is provided a method for detecting the presence of gynaecological cancer according to the invention, wherein the antibodies used under step (ii) is a combination of antibodies wherein the combination comprises (a) an antibody against exon 1 from full length BARD 1 (SEQ ID NO: 12); (b) an antibody against exon 4 from full length BARD 1 (SEQ ID NO: 12); and (c) an antibody exon 11 from full length BARD 1 (SEQ ID NO: 12).

According to another further aspect of the invention, is provided a method for detecting the presence of gynaecological cancer according to the invention, wherein an antibody against exon 4 from full length BARD 1 (SEQ ID NO: 12) is an antibody against a polypeptide of SEQ ID NO: 25.

According to another further aspect of the invention, is provided a method for detecting the presence of gynaecological cancer according to the invention, wherein the antibodies used under step (iii) is a combination of antibodies wherein the combination comprises (a) at least one antibody against exon 1 from full length BARD 1 (SEQ ID NO: 12); (b) at least one antibody against exon 7 from full length BARD 1 (SEQ ID NO: 12); and (c) at least one antibody against exon 10 and/or exon 11 from full length BARD 1 (SEQ ID NO: 12).

A method according to any one of claims 11 to 15 and 21, wherein the antibodies used under step (iii) is a combination of antibodies wherein the combination comprises (a) at least one antibody against exon 1; (b) at least one antibody against exon 7; and (c) at least one antibody against exon 11 from full length BARD 1 (SEQ ID NO: 12).

According to another further aspect of the invention, is provided a method for detecting the presence of gynaecological cancer according to the invention, wherein the specific antibodies used under step (ii) is a combination of antibodies wherein the combination comprises (a) at least one antibody against exon 1 from full length BARD 1 (SEQ ID NO: 12; and at least one antibody against a sequence selected from the following group: SEQ ID NO: 21 and SEQ ID NO: 22.

According to another further aspect of the invention, is provided a method according to the invention, wherein the biological sample is isolated from a human subject.

According to another further aspect of the invention, is provided a method according to the invention, wherein the biological sample is blood.

According to another further aspect of the invention, is provided a method according to the invention, wherein the steps (b) and/or (c) in any one of claims 1 to 10 or the detection steps (iv) and/or (v) in any one of claims 23 to 25, are assayed for with an assay selected from an ELISA assay and a western blotting assay.

According to another further aspect of the invention, is provided a method according to the invention, wherein the comparison step (b) or the detection under step (iv) are assayed for with an assay selected from an ELISA assay wherein the biological sample is a blood sample.

According to another aspect of the invention, is provided an isolated polypeptide comprising at least one sequence of amino acids having at least 80% identity or homology (such as at least 85%, at least 90%, at least 95%, at least 98%) with a sequence of amino acids selected from SEQ ID NO: 2, SEQ ID NO: 3, SEQ ID NO: 4, SEQ ID NO: 5, SEQ ID NO: 6, SEQ ID NO: 7; SEQ ID NO: 8; SEQ ID NO: 9; SEQ ID NO: 10, SEQ ID NO: 11 and SEQ ID NO: 24.

According to a further aspect of the invention, is provided an isolated polypeptide according to the invention, having a sequence of amino acids having at least 80% identity or homology (such as at least 85%, at least 90%, at least 95%, at least 98%) with a sequence of amino acids selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9.

According to another further aspect of the invention, is provided an isolated polypeptide according to the invention, having a sequence of amino acids having at least 80% identity or homology (such as at least 85%, at least 90%, at least 95%, at least 98%) with a sequence of amino acids selected from SEQ ID NO: 10; SEQ ID NO: 11 and SEQ ID NO: 24.

According to another further aspect of the invention, is provided an isolated polypeptide according to the invention, having a sequence of amino acids selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9.

According to another further aspect of the invention, is provided an isolated polypeptide according to the invention, having a sequence of amino acids selected from SEQ ID NO: 10, SEQ ID NO: 11 and SEQ ID NO: 24.

According to another aspect of the invention, is provided an isolated nucleic acid consisting of a nucleotide sequence encoding a polypeptide according to the invention.

According to a further aspect of the invention, is provided an isolated nucleic acid consisting of a nucleotide sequence according the invention selected from the group consisting of SEQ ID NO: 13; SEQ ID NO: 14; SEQ ID NO: 15; SEQ ID NO: 16; SEQ ID NO: 17; SEQ ID NO: 18, SEQ ID NO: 19; SEQ ID NO: 20 and SEQ ID NO: 23.

According to another aspect of the invention, is provided a use of a nucleic acid according to claim 33 or 34 for expressing recombinant polypeptides for analysis, characterization and therapeutic use.

According to a further aspect of the invention, is provided a use of a nucleic acid according to the invention as probes or primers.

According to another aspect of the invention, is provided an isolated antibody that selectively binds at least one polypeptide according to the invention.

According to a further aspect of the invention, is provided an isolated antibody according to the invention that selectively binds at least one polypeptide according to the invention.

According to another aspect of the invention, is provided a combination of antibodies comprising (a) at least one antibody against exon 1 from full length BARD 1 (SEQ ID NO: 12); (b) at least one antibody against one exon selected from exon 4, exon 5, exon 6, exon 7, exon 8 and exon 9 from full length BARD 1 (SEQ ID NO: 12); and (c) at least one antibody against exon 10 and/or exon 11 from full length BARD 1 (SEQ ID NO: 12).

According to a further aspect of the invention, is provided a combination of antibodies according to the invention comprising (a) at least one antibody against exon 1 from full length BARD 1 (SEQ ID NO: 12); (b) at least one antibody against one exon selected from exon 4, exon 5 and exon 6 from full length BARD 1 (SEQ ID NO: 12); and (c) at least one antibody against exon 10 and/or exon 11 from full length BARD 1 (SEQ ID NO: 12).

According to another further aspect of the invention, is provided a combination of antibodies according to the invention comprising (a) at least one antibody against exon 1 from full length BARD 1 (SEQ ID NO: 12); (b) at least one antibody against exon 4 from full length BARD 1 (SEQ ID NO: 12); and (c) at least one antibody against exon 10 and/or exon 11 from full length BARD 1 (SEQ ID NO: 1).

According to another further aspect of the invention, is provided a combination of antibodies according to the invention comprising (a) an antibody against exon 1 from full length BARD 1 (SEQ ID NO: 12); (b) an antibody against exon 4 from full length BARD 1 (SEQ ID NO: 12); and (c) an antibody exon 11 from full length BARD 1 (SEQ ID NO: 12).

According to another further aspect of the invention, is provided a combination of antibodies according to the invention, wherein the antibody against exon 4 from full length BARD 1 (SEQ ID NO: 12) is an antibody against a polypeptide of SEQ ID NO: 25.

According to another aspect of the invention, is provided a combination of antibodies comprising (a) at least one antibody against exon 1 from full length BARD 1 (SEQ ID NO: 12); (b) at least one antibody against exon 7 from full length BARD 1 (SEQ ID NO: 12); and (c) at least one antibody against exon 10 and/or exon 11 from full length BARD 1 (SEQ ID NO: 12).

According to another further aspect of the invention, is provided a combination of antibodies according to the invention comprising (a) at least one antibody against exon 1; (b) at least one antibody against exon 7; and (c) at least one antibody against exon 11 from full length BARD 1 (SEQ ID NO: 12).

According to another aspect of the invention, is provided a combination of antibodies comprising at least one antibody against exon 1; and at least one antibody against a sequence selected from the following group: SEQ ID NO: 21 and SEQ ID NO: 22.

According to another aspect of the invention, is provided a method for detecting the level of cellular expression of proteins of comprising the step of:

(i) Contacting at least one antibody according to the invention or a fragment thereof, or a combination of antibodies according to the invention with cells to be tested under appropriate conditions for binding of the said antibodies, combination thereof or combination of antibodies to at least a protein having a sequence of amino acids selected from SEQ ID NO: 2, SEQ ID NO: 3, SEQ ID NO: 4, SEQ ID NO: 5, SEQ ID NO: 6, SEQ ID NO: 7; SEQ ID NO: 8; SEQ ID NO: 9; SEQ ID NO: 10, SEQ ID NO: 11 and SEQ ID NO: 24 on the cells;
(ii) Determining the level of binding of said at least one antibody, combination thereof, or combination of antibodies to the cell as indicative level of expression of the said protein.

According to another aspect of the invention, is provided a use of an antibody according to the invention or a combination of antibodies according to the invention in an assay.

According to a further aspect of the invention, is provided a use according to the invention wherein the assay is western blots, immunohistochemistry, ELISA or FACS assays.

According to a further aspect of the invention, is provided a use of an antibody according to the invention or a combination of antibodies according to the invention in a method according to the invention.

According to another aspect of the invention, is provided a recombinant expression vector comprising a nucleic acid molecule according to the invention, wherein the vector optionally comprises an expression control sequence, allowing expression in prokaryotic or eukaryotic host cells of the encoded polypeptide, operably linked to the nucleic acid molecule.

According to another aspect of the invention, is provided a host cell transfected or transformed with a recombinant expression vector according to the invention or a nucleic acid according to the invention.

According to another aspect of the invention, is provided a process for producing cells capable of expressing a polypeptide according to the invention comprising genetically engineering cells with a vector according to the invention or a nucleic acid according to the invention.

According to another aspect of the invention, is provided a kit comprising at least one polypeptide according to the invention.

According to another aspect of the invention, is provided an immunoassay kit for detecting gynaecological cancer in a biological sample, the kit comprising at least one antibody according to the invention or a fragment thereof or a combination of antibodies according to the invention.

According to another aspect of the invention, is provided a use of an antagonist of a polypeptide according to the invention for the manufacture of a medicament for the treatment of a gynaecological cancer, including breast, ovarian, cervical and uterine cancers. In a particular embodiment, the antagonist is an antibody or a combination of antibodies according to the invention.

According to another aspect of the invention, is provided a method of treating a disease comprising the administration of a therapeutically effective amount of an antagonist of a polypeptide according to the invention in a mammal in need thereof; wherein the disease is a gynaecological cancer, including breast, ovarian, cervical and uterine cancers.

The BARD1 isoforms, polypeptides and antibodies of the invention may be useful in the Prognostic and diagnostic of gynaecological cancers

The N-terminus of BARD1 interacts with BRCA1, and is essential for BARD1's well established tumour suppressor function. Therefore loss of the N-terminus, such as observed in the omega isoforms, correlates with loss of tumour suppressor function. In the absence of further mutations, it is possible that the cell would not be a cancer cell.

However, absence of the BARD1 N-terminus, such as observed in the omega isoforms, is an indication of a predisposition to develop a cancer because of the absence of an important tumour suppressor function. Detection of BARD1 forms lacking the N-terminus such as omega iso forms or more generally iso forms with a start in exon 3 or downstream of exon 3 or forms of N-terminally proteolytically cleaved BARD1 can be used as a predictive tool to establish predisposition to a cancer. Specifically, detection of omega isoforms is predictive of a high risk of developing a gynaecological cancer. Moreover, in many cases, at the time of testing, a cancer will already have developed in the absence of this tumour suppressor function, and the detection of omega iso forms will correlate in these cases with the presence of a gynaecological cancer. Consequently, if omega isoforms are detected in a patient, further investigation will be appropriate to establish whether the patient already has a gynaecological cancer. If the patient is found not to have a cancer at the time of initial testing, then the patient will have to be closely monitored to detect the appearance of a gynaecological cancer rapidly after its event.

The exons in the middle part of BARD1, such as observed in the splice isoforms (alpha, beta and more importantly phi, delta, epsilon and eta), are important for the well established tumor suppressor function together with BRCA1 residing in exons 2 and the apoptotic function of BARD1 residing in exons 5 through 8. Therefore loss of exons in this region, such as observed in the splice iso forms gives BARD1 proliferation-inducing properties, making it oncogenic on its own. Therefore, absence of exons in the middle part of BARD1 is indicative of the presence of a gynaecological cancer. Absence of such splice isoforms, however, is not indicative of the absence of a cancer. Detection of splice iso forms can be used as a diagnostic tool to establish the presence of a gynaecological cancer.

BARD1-based diagnostic screening for gynaecological cancers or high risk of developing such cancers will in any case have to be undertaken in combination with other diagnostic methods as gynaecological cancers could, in some cases, also occur without expression of BARD1 splice or omega isoforms.

References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

The invention having been described, the following examples are presented by way of illustration, and not limitation.

### EXAMPLES

The following abbreviations refer respectively to the definitions below:

kDa (Kilo Dalton), μg (microgram), μl (microliter), min (minute), mM (millimolar), sec (second), BRCA1 (Breast cancer 1), BSA (bovine serum albumin), CCC (clear cell carcinoma), CIP (calf intestinal phosphatase), DAB (diaminobenzidine), DAPI (4′,6-diamidino-2-phenylindole), EDTA (Ethylenediaminetetraacetic acid), EnC (Endometriod carcinoma), FL (Full length), GFP (Green Fluorescent Protein), HRP (horse radish peroxidase), LNA (Nitro(imidazole/triazole)-linked acridine), MuC (mucinous carcinoma), MLV (Murine Leukemia virus), MW (molecular weight), ORF (Open reading frame), PNA (peptide nucleic acid), RT-PCR (reverse transcriptase polymerase chain reaction), SDS (Sodium Dodecyl Sulfate), SeC (serous carcinoma), siRNA (small interfering RNA), TAE (Tris-acetate-EDTA), TBS (Tris buffered saline), TNM (Tumor Node Metastatis), UV (ultraviolet), WFS (Anti-BARD1 antibody WFS).

### Example 1

### Structure of BARD1 Isoforms

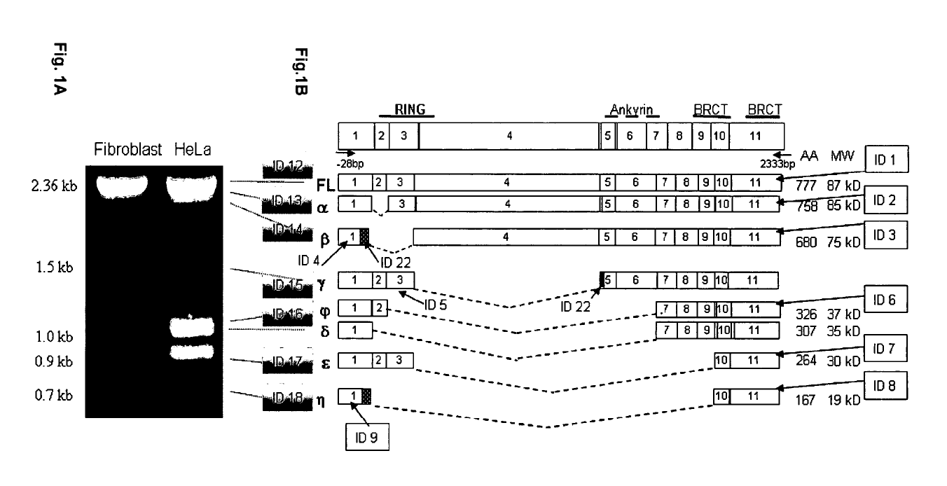

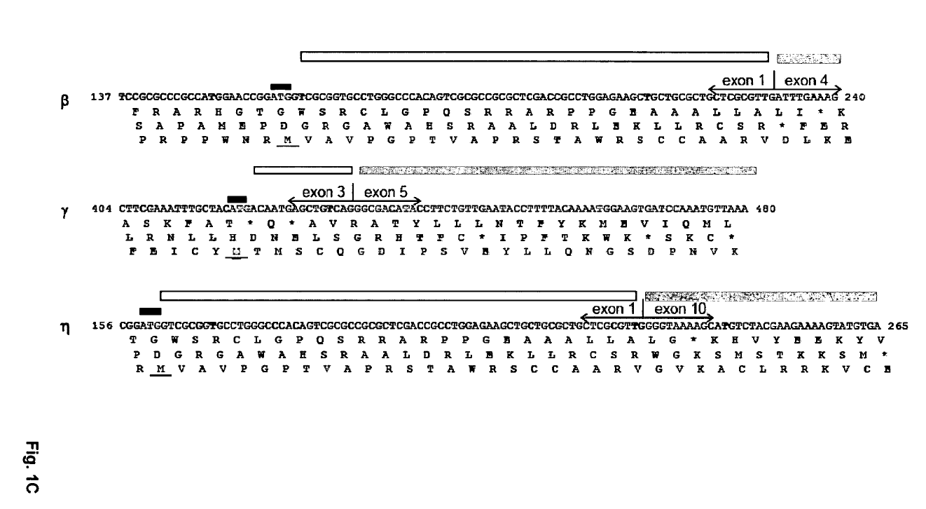

To unravel the expression pattern of BARD1, the structure of BARD1 isoforms was determined in human normal fibroblasts and in Hela cells by RT-PCR. BARD1 was highly expressed in normal fibroblasts, and there was almost no expression of BARD1 iso forms when primers for amplifying the entire coding region were used. In Hela cells, spliced isoforms of BARD1 were highly expressed together with FL BARD1 (FIG. 1A). These iso forms were cloned and sequenced and their structure, exon composition, and calculated molecular weight (MW) were determined (FIG. 1B). FL BARD1 (SEQ ID NO: 12) translates into a protein of 777 amino acids or a calculated MW of 87 kDa (SEQ ID NO: 1).

Isoform α has a deletion of exon 2 (SEQ ID NO:13) and produces a 85 kDa protein of 758 amino acids (SEQ ID NO: 2). Isoform β, derived from deletion of exon 2 and 3 (SEQ ID NO:14), translates into a protein of 680 amino acid or 75 kDa, but would use a translation start in an alternative reading frame of exon 1 (SEQ ID NO: 3 and SEQ ID NO: 4). Deletion of exon 4 in isoform γ (SEQ ID NO:15) disrupts the open reading frame. However, isoform φ and δ, missing exons 3 to 6 (SEQ ID NO:16) or 2 to 6, could produce a 37 or 35 kDa protein of 326 amino acids (SEQ ID NO: 6) or 307 amino acids; only δ was reported previously in HeLa (Tsuzuki et al., 2005, above) and ovarian cancer cells (Feki et al., 2005, above); and isoform ε lacking exons 4 to 9 (SEQ ID NO:17) with a predicted MW of 30 kDa, composed of 264 amino acids (SEQ ID NO:7), and isoform η is composed of exons 1, 10, and 11 (SEQ ID NO:18), which is not in frame but initiation of translation could occur in an alternative reading frame and translate into a 19 kDa protein of 167 amino acids (SEQ ID NO: 8 and SEQ ID NO: 9). All these iso forms might loose either the RING domain or the ANK and BRCT domain, which are the important regions for BARD1 function as a tumour suppressor, and consequently lead to a tumourogenesis function.

### Example 2

### Expression of BARD1 in Different Cancer Cell Lines

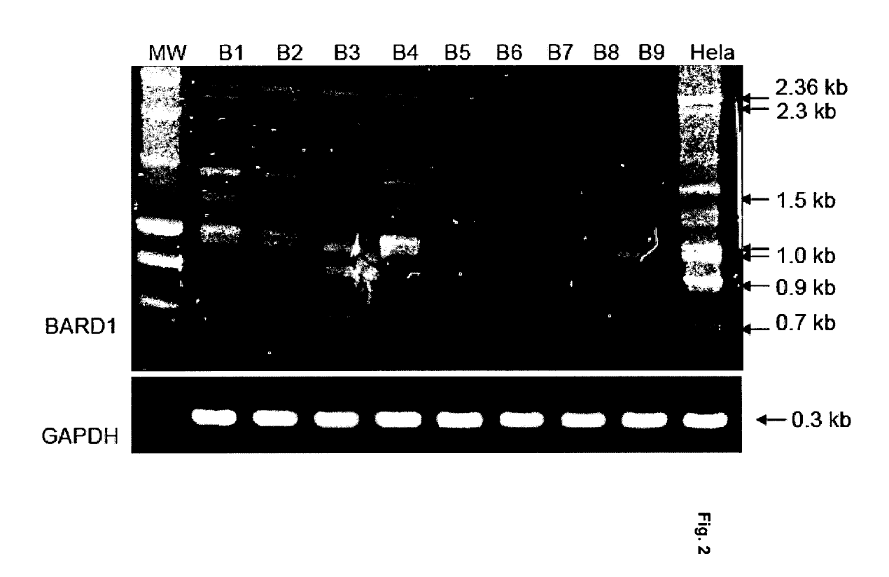

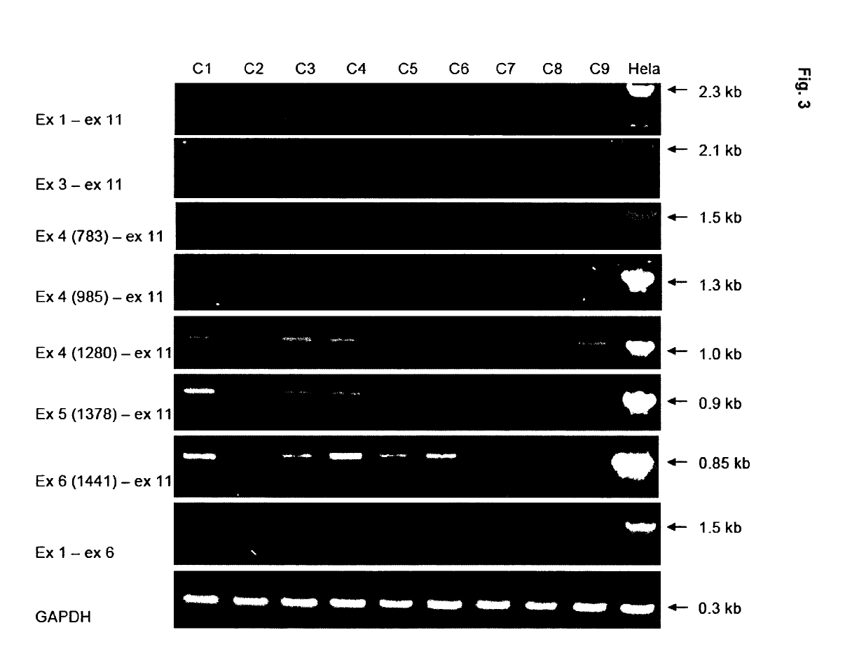

To further investigate the structure of BARD1 isoforms, RT-PCR was performed on RNA from different gynaecological cancer cell lines to characterize BARD1 expression. Primers located in various exons of BARD1 were used to amplify different regions of BARD1 for breast, cervical, endometrial, and ovarian cancer cell lines. A specific BARD1 expression pattern in cell lines derived from different cancers was observed. Firstly, in breast cancer cell lines FL BARD1 was expressed together with smaller isoforms: β, φ, δ, and ε which were more abundant than FL BARD1. Another group showed no expression at all when primers were used for amplification of FL BARD1 (FIG. 2).

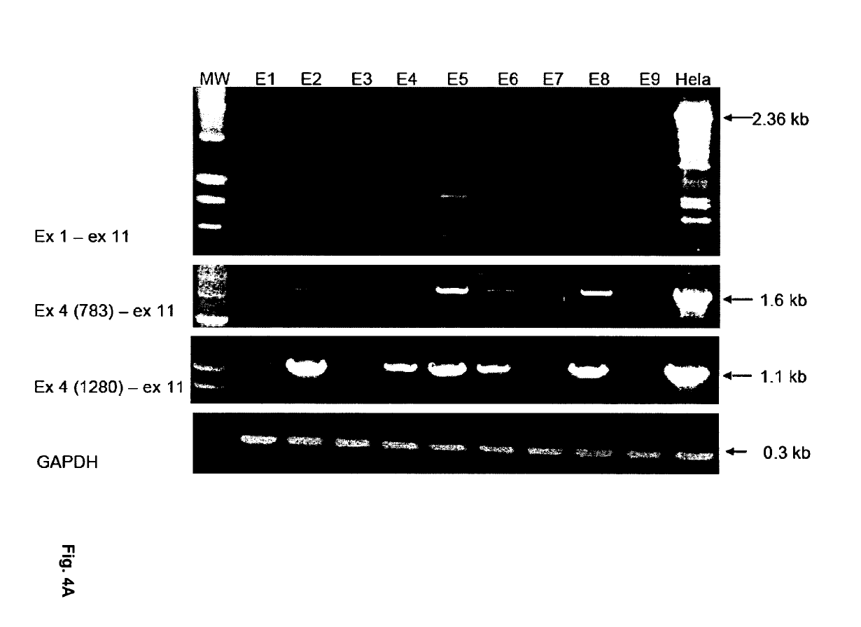

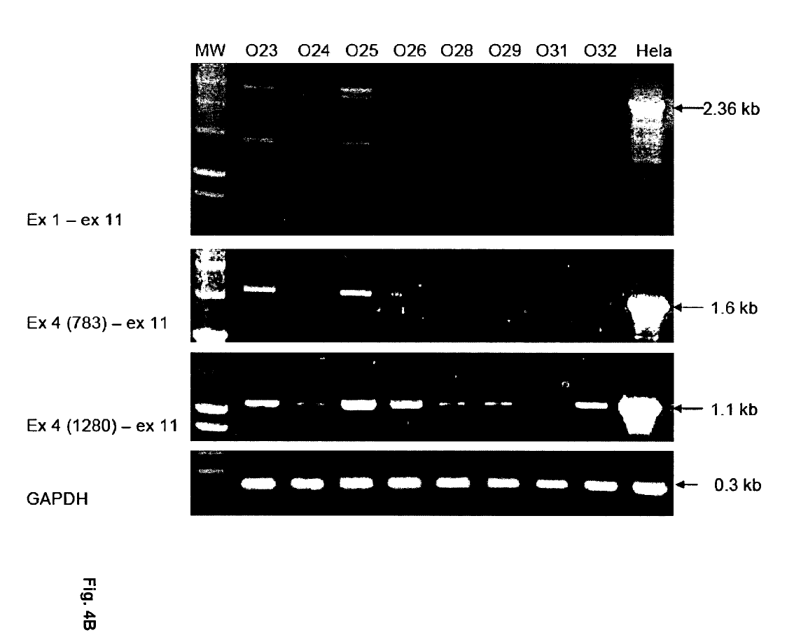

In all cervical cancer lines, neither FL BARD1 nor splice isoforms were found, when RT-PCR was performed to amplify exon 1 to exon 11. Different forward primers more downstream were then used to amplify potentially 5′ truncated forms of BARD1, and BARD1 expression was detected when using primers at different sites in exon 4. Finally, BARD1 expression was found in all samples when forward primer in exon 5 (FIG. 3) was used. It seems that these BARD1 isoforms were initiated in exon 4 in cervical cancer cell lines. Two new transcription initiations were found within exon 4 for ovarian cancer. One was at the nucleotide position of 458 (start 1), which was at the beginning of exon 4 and the other was at the 983 nucleotide (start 2) in exon 4. In Hela cells, the new transcription initiation located at the end of exon 4, at nucleotide position 1290 (start 3). Start 1 and 2 transcript at the same ATG within exon 4 and produce a protein of approximately 44 kDa, and start 3 could produce a protein about 27 kDa. The mRNA and translated sequence structure was shown in FIG. 5B. The new isoforms initiating in exon 4 were named Ω1, Ω2 and Ω3. In our RT-PCR experiments, the forward primer within exon 4 at nucleotide position 783 detected isoform Ω1 (SEQ ID NO: 10), and primes at nucleotide position 985 and 1280 detected isoform Ω2 (SEQ ID NO: 11. Isoform Ω3 (SEQ ID NO: 24) could be detected by forward primer within exon 5 at nucleotide position 1378.

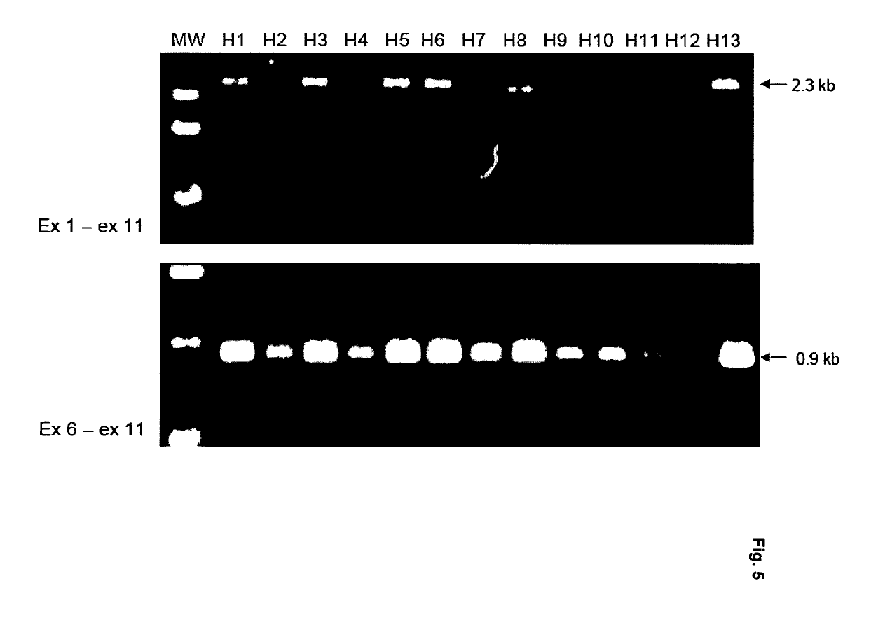

RT-PCR was performed in endometrial cancer and ovarian cancer cell lines by using forward primers within exon 4 (FIGS. 4A and B). FL BARD1 and isoforms were expressed in some of the samples. In other samples, which showed neither FL BARD1 nor isoforms, BARD1 was detected by forward primers within exon 4.

In summary (Table 2 below), using RT-PCR in breast cancer either little or no FL was observed, whereas splice isoforms or only omega iso forms were observed. In cervical cancer, only omega isoforms were observed, in endometrial and ovarian cancer, either little or no FL was observed but rather the presence of splice isoforms. Endometrial and ovarian cancer lines also expressed omega isoforms. By Western Blot, very little or no FL, expression of all splice iso forms and of omega iso forms were observed in ovarian cancer. Overall, in all gynaecological cancers there is little or no FL BARD1, but rather the expression of splice and/or omega isoforms was observed. In all cases, when FL and iso forms were expressed, FL was much less abundant than the iso forms.

TABLE 2

| | | SPLICE | OMEGA |
|---|---|---|---|
| CANCER TYPE | FL BARD 1 | ISOFORMS | ISOFORMS |
| CERVICAL | 0 | 0 | +++ |
| BREAST | + | ++ | 0 |
| | 0 | 0 | +++ |
| OVARIAN | + | ++ | 0 |
| (Based on WB data) | 0 | 0 | +++ |
| | + | ++ | +++ |
| ENDOMETRIAL | + | ++ | 0 |
| | 0 | 0 | +++ |
| LYMPHOMA | ++ | (+) | 0 |
| LUNG CANCER | 0 | 0 | 0 |
| NORMAL CELLS | + | 0 | 0 |
| (CONTROL) | | | |

By contrast, in lymphoma where FL and splice iso forms are expressed, FL is much more abundant than splice isoforms. In lung cancer, none of FL BARD1, omega isoforms or splice isoforms (as those seen in gynaecological cancers) was observed. Expression of splice isoforms is characteristic of gynaecological cancers, and non-gynaecological cancers either do not express any splice isoforms, or splice isoforms are expressed at very low levels, and always much less abundant than FL BARD1.

The relative prevalence of the different patterns observed in gynaecological cancers is indicated in Table 3 below. In none of the cervical cancer cell lines tested was the expression of FL BARD1 observed, and only BARD1 omega isoforms were present. In endometrial cancer, FL and isoforms were expressed in 55.6% of the cases, in 11.1% of the cases only spliced isoforms were present, and 33.3% of the cases showed omega isoforms. In breast cancer cells, 19.2% of the cases expressed FL BARD1 and isoforms, and most of the cell lines expressed omega isoforms, which accounted for about 80.8%. In ovarian cancer cell lines, 21.9% expressed FL and isoforms, 15.6% expressed spliced isoforms only, and 62.5% expressed transcripts comprising exon 4 through exon 11. All the tested cancer cell lines were derived from cancers which might be hormonally regulated. In all of the gynaecological cancer cell lines tested, FL BARD1 was either missing or it seemed less abundant than other smaller isoforms. All the cancer cell lines expressed one or the other form of BARD1.

As a comparison, RT-PCR was also performed in haematological tumour cell lines which are unlikely to be hormonally controlled. Thus it was concluded that FL BARD1 is often lost in gynaecological cancer cell lines, but instead either splice isoforms or omega iso forms are expressed.

TABLE 3

| | | FULL | | |
|---|---|---|---|---|
| | FULL | LENGTH | SPLICE | |
| TYPE OF | LENGTH | & SPLICE | ISOFORMS | OMEGA |
| CANCER | ONLY | ISOFORMS | ONLY | ISOFORMS |
| Cervical | 0 | 0 | 0 | 100% (9/9) |
| cancer | | | | |
| Breast | 0 | 19.2% (5/26) | 0 | 80.8% (21/26) |
| cancer | | | | |
| Ovarian | 0 | 21.9% (7/32) | 15.6% (5/32) | 62.5% (20/32) |
| cancer | | | | |
| Endometrial | 0 | 55.6% (5/9) | 11.1% (1/9) | 33.3% (3/9) |
| cancer | | | | |
| Lymphoma | 61.5% (8/13) | 38.5% (5/13) | 0 | Not determined |

From these results the following could be deduced:

In many cases, gynaecological cancers (breast, ovarian, endometrial or cervical) express splice isoforms, always in much higher abundance than FL BARD1. In some cases, gynaecological cancers (breast, ovarian, endometrial or cervical) express omega isoforms but no FL and no splice isoforms. In some cases, gynaecological cancers (breast, ovarian, endometrial or cervical) express both splice and omega isoforms. However, in lung cancer no FL BARD1 or the splice isoforms observed in gynaecological cancers or the omega iso form have been observed. Normal cells only express FL BARD1. Lymphoma cell lines express FL BARD1 and sometimes splice isoforms, but these are always in much lower abundance than FL BARD1.

### Example 3

### Protein Expression Patterns of BARD1 Isoforms (Detected by Western Blot or ELISA)

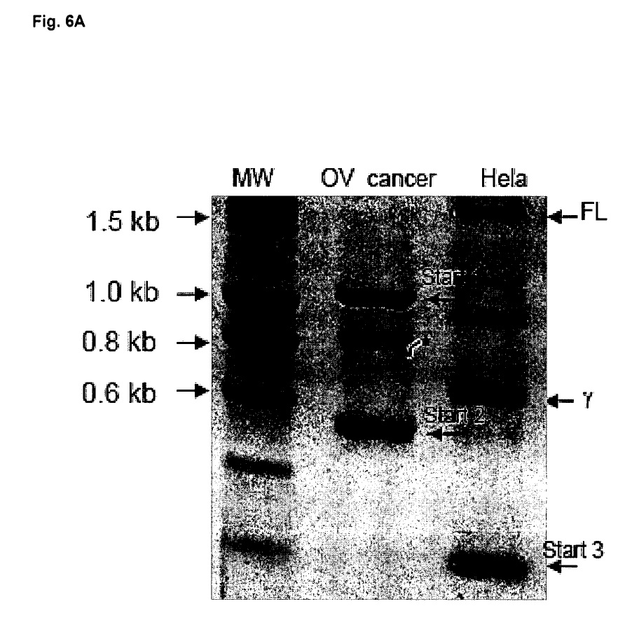

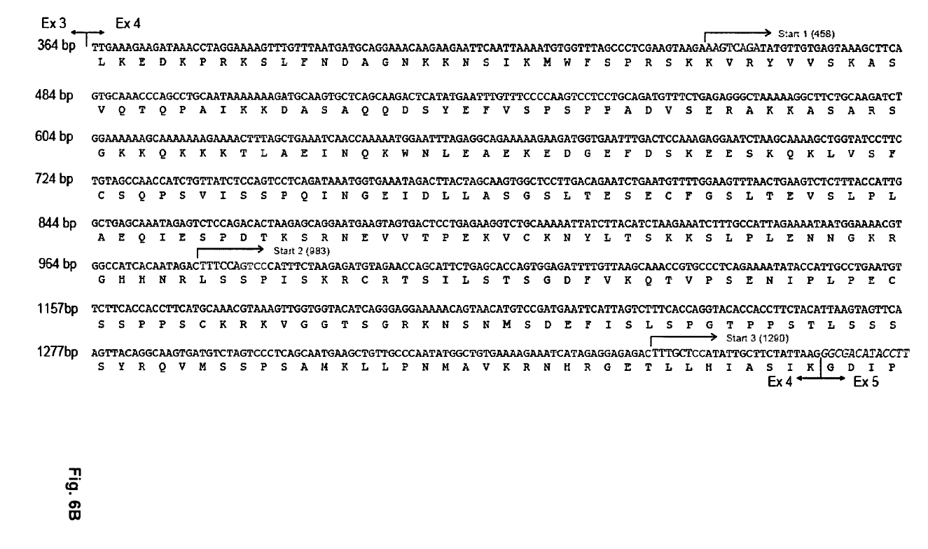

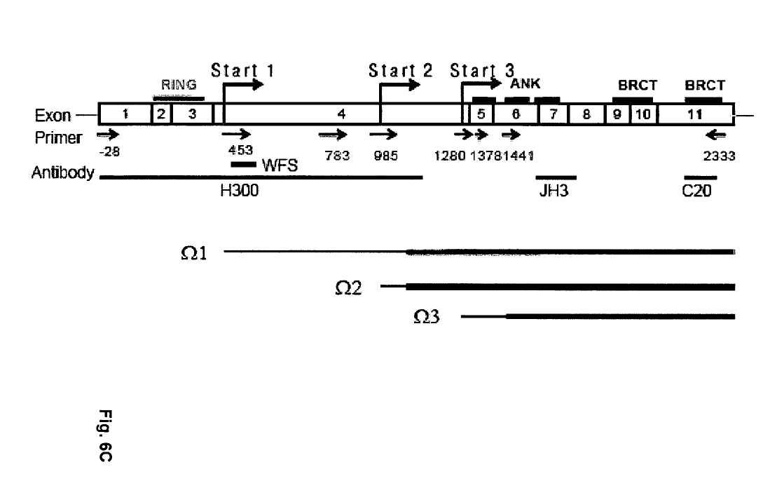

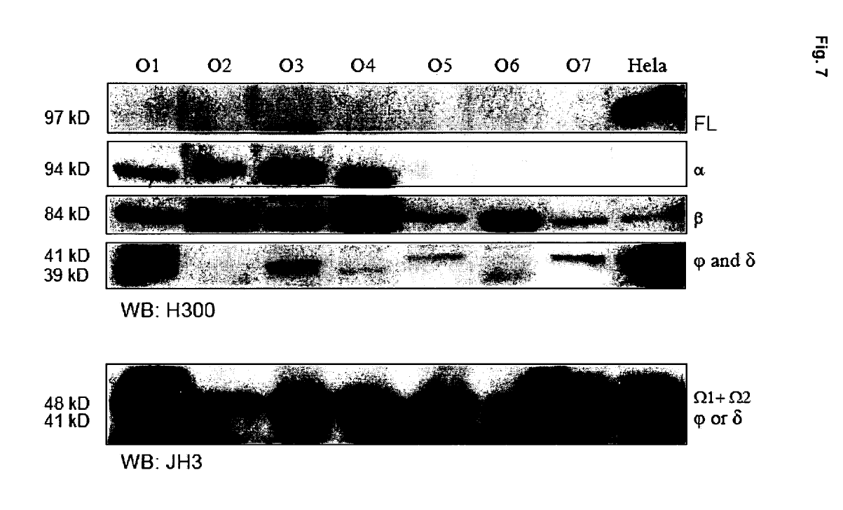

Splice iso forms could for example be detected with a combination of antibodies against exon 1 (such as for example antibody N19), exon 4 (e.g. antibody WSF), and exon 11 (e.g. antibody C20) (FIG. 6).

The antibody against exon 1 recognizes FL BARD1 and all splice isoforms but not omega isoforms. The antibody against exon 11 recognizes FL BARD1, all splice isoforms and all omega isoforms. The antibody against exon 4 recognizes FL BARD1 and does not recognize splice isoforms.

In another embodiment, the above antibody against exon 4 would be directed against the sequence LKEDKPRKSLFNDAGNKKNSIKMWFSPRSK (SEQ ID NO: 25) located at the beginning of exon 4. Such an antibody would recognize only FL BARD1 but not splice isoforms or omega isoforms. It would recognize isoform beta.

Another possibility for detecting splice iso forms would be to use an antibody directed against the sequence MVAVPGPTVAPRSTAWRSCCAARV (SEQ ID NO: 21) which is characteristic of the beta and eta splice isoforms expressed from an alternative reading frame. This sequence is only present in beta and eta and allows their identification without cross-reaction with FL BARD1. Beta and eta are usually expressed together with other splice isoforms, so their presence would be indicative of expression of splice isoforms in general. Antibodies against the sequence GRHTFC (SEQ ID NO: 22) in the gamma splice isoform could achieve the same purpose. Alternatively, one could use an antibody directed against exon 7 (e.g. antibody JH3, see FIG. 6), which would recognize all omega isoforms as well as the splice isoforms alpha, beta, phi and delta but not epsilon and eta. All the antibodies directed against exons 4, 5, 6 or 7 would also recognize FL BARD1. It would be a matter of calibrating the signal ratios to determine which pattern is being recognized.

### 1-4-11 Combination (N19-WSF-C20):

FL would give 1high-4-high-11high

Splice isoforms would give 1high-4very low-11high

Omega isoforms would give 1null-4very low/null-11high

Little FL and more splice would give 1high-4low-11high

Little splice and more FL would give 1high-4medium/high-11high

### Read-Out of the Above 1-4-11 Test:

If 1 is lower than 11, then there are omega isoforms expressed, which is predictive of an increased risk of developing a gynaecological cancer.

If 4 is lower than 1, then there are splice iso forms expressed, which is indicative of the presence of a cancer.

If 4 is lower than 1 and 4 is low, then splice iso forms are present and more abundant than FL, which would be indicative of the presence of a gynaecological cancer.

### 1-7-11 Combination (N19-JH3-C20):

FL would give 1high-Thigh-11high

Splice isoforms would give 1high-7low-11high

Omega isoforms would give 1null-Thigh-11high

Little FL and more splice would give 1high-7low/medium-11high.

### Example 4

### Identification of BARD1 Protein Isoforms in Ovarian Cancer Cell Lines

As different BARD1 transcripts were observed in cancer cells, it has been investigated whether these isoforms were translated. Western Blot analysis was performed on protein extracts from ovarian cancer cell lines. Hela cells were used as control. BARD1 antibody H300 against epitopes expressed on exon 1 through 4, and antibody JH3 directed against a peptide antigen within exon 7 for C terminal, were used. FIG. 7 shows how it would be possible to detect FL, splice isoforms and omega isoforms in the same sample by Western blot. Individual iso forms could be identified through a combination of reactivity with a specific antibody and size on the gel.

When using H300, we found that FL BARD1, which migrates on the gel as a band of 97 kDa was detected in extracts from Hela cells, but none of the ovarian cancer samples showed the FL BARD1. We detected protein bands of 94 kDa, 84 kDa and 68 kDa in all these cases. Concluding from the structure for the mRNA expressed in ovarian cancer, the 94 kDa and 84 kDa bands corresponded to isoform α (deletion exon 2) and iso form β (deletion exon 2 and 3), respectively. The 68 kDa band remains unknown. In some of the samples, several smaller bands of 40 to 50 kDa were observed, which were weakly expressed. However, when probing with JH3, a very strong band of 48 kDa was detected, which was barely detected by H300 (FIG. 7). This N-terminally truncated form was abundantly expressed in ovarian cancer samples. The observed MW of this protein corresponds to the calculated MW of isoforms Ω1 and Ω2, which was about 44 kDa when migration on gel slightly higher like FL BARD1 could account for 48 kDa. This 48 kDa protein could derive from isoforms Ω1 and Ω2 (SEQ ID NO: 10 and SEQ ID NO: 11), which is consistent with our RT-PCR result. It is also deduced that the other smaller band of about 41 kDa detected by JH3 could be isoform φ (deletion exon 3 to 6) or δ (deletion exon 2 to 6). The result of Western blots thus confirmed the results obtained by RT-PCR and provided evidence that there was little or no FL BARD1 expressed in ovarian cancer, but instead different splice and omega isoforms were expressed. Compared to the splice isoforms, isoforms Ω1 and 2 were most abundant. This figure shows that, at protein level, both splice and omega iso forms but no FL BARD1 are detectable in ovarian cancer cell lines.

### Example 5

### BARD1 Expression in Ovarian Cancer Patients

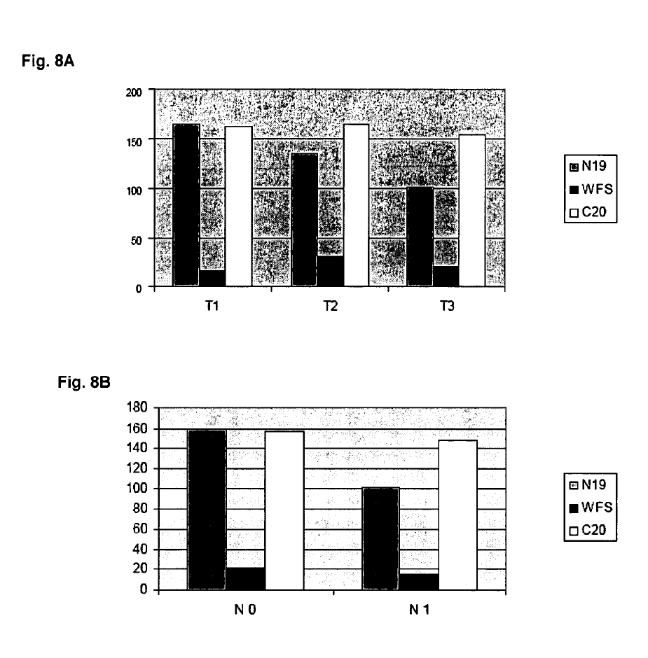

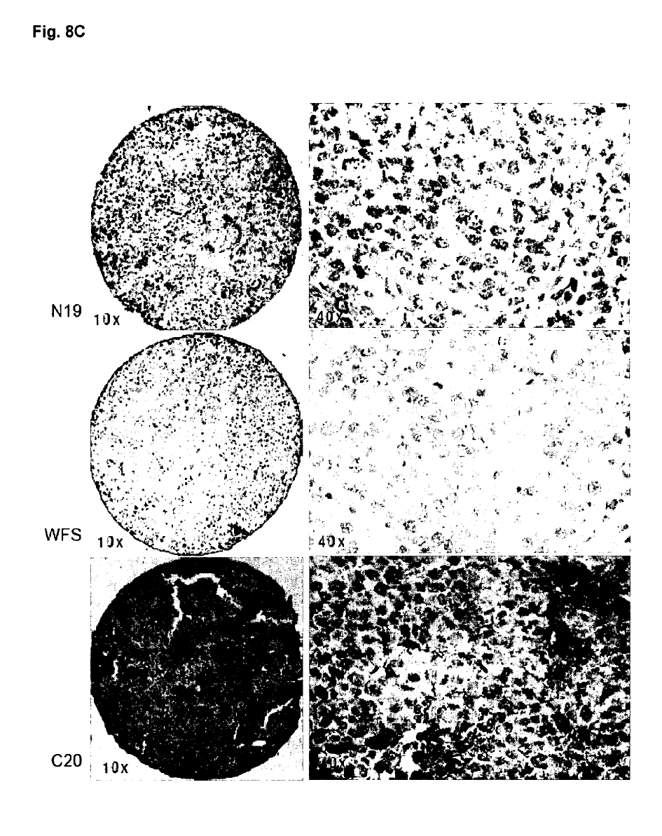

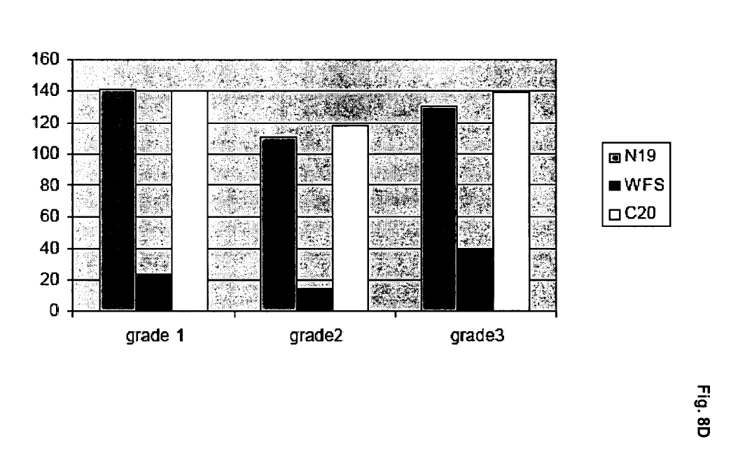

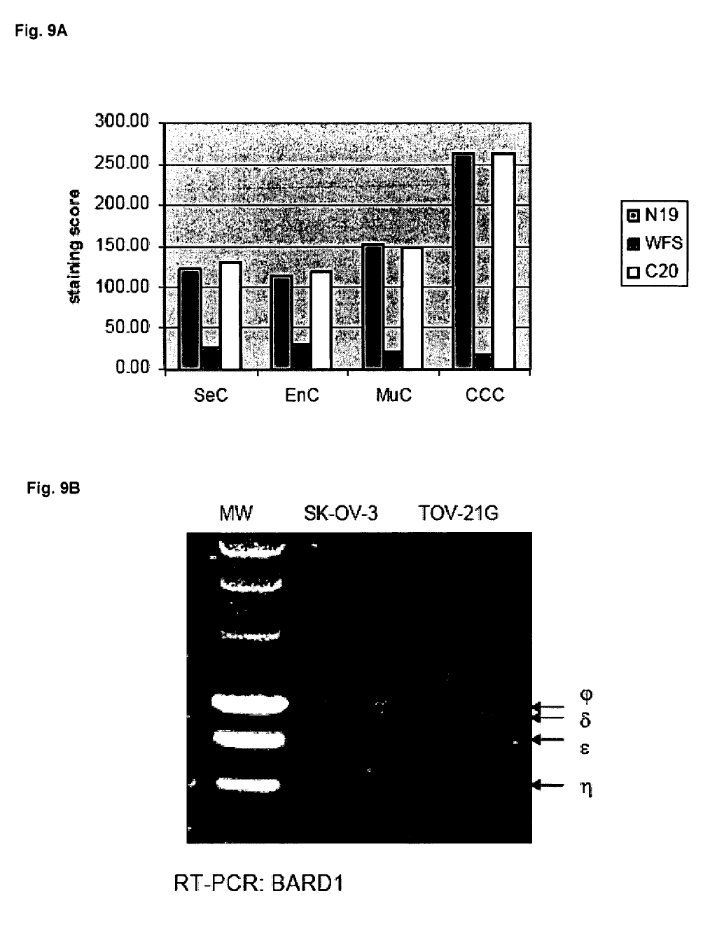

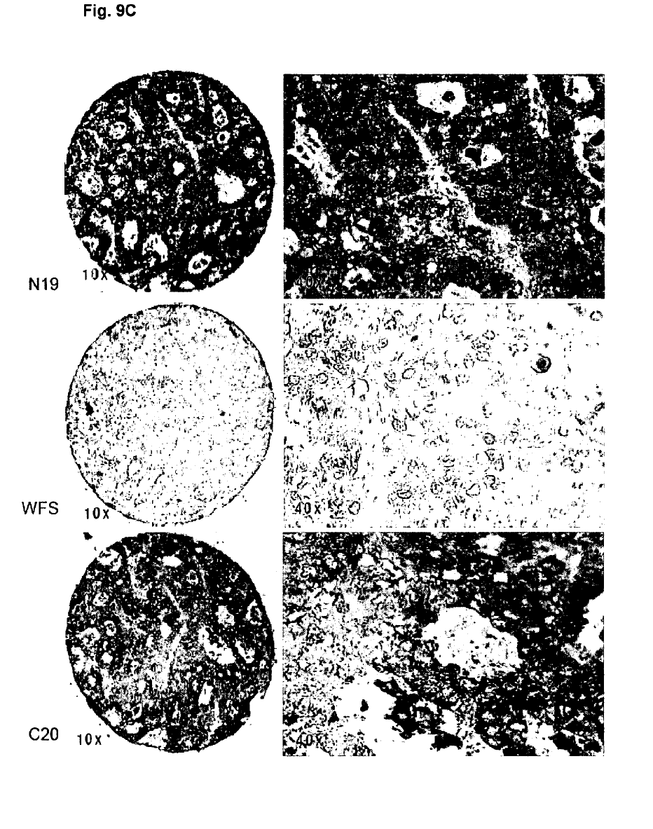

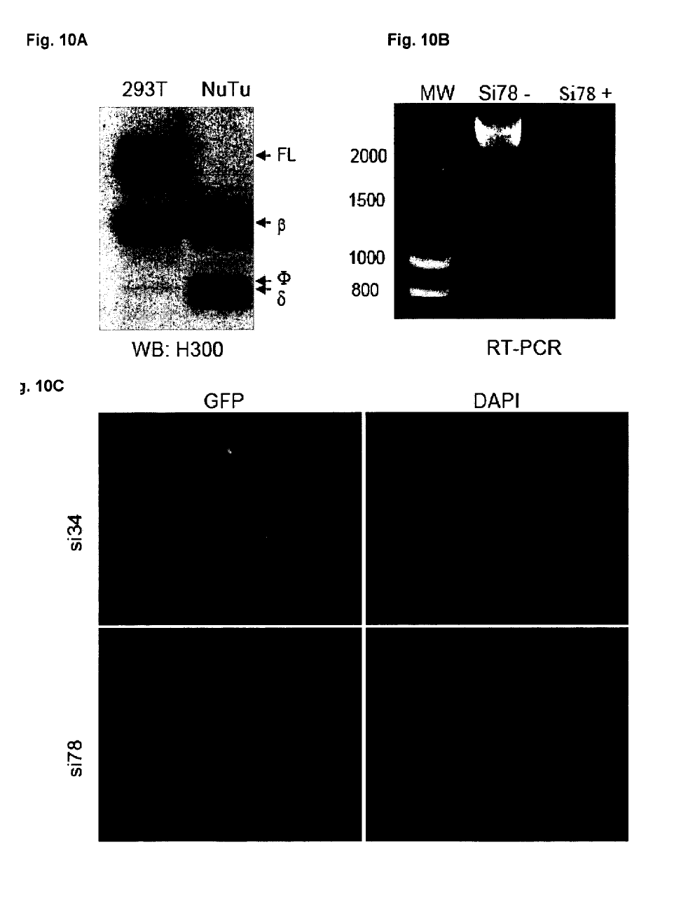

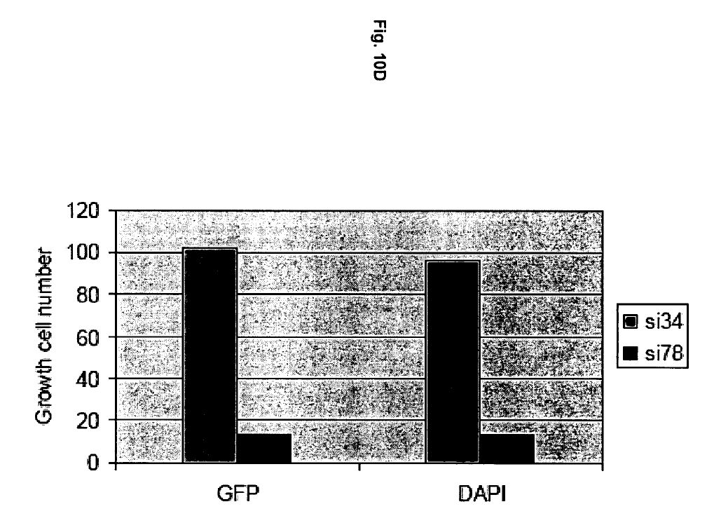

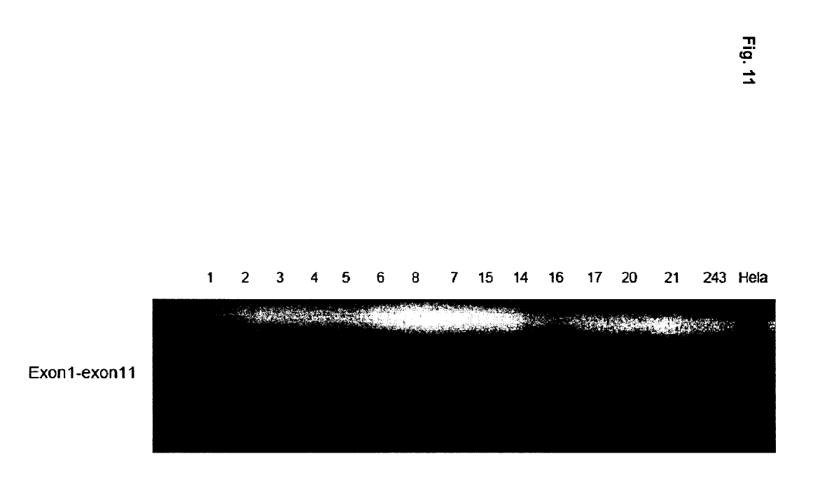

To investigate how BARD1 was expressed and correlated with carcinogenesis and cancer progression, immunohistochemical staining was performed on a tissue array of ovarian cancers. Different antibodies detecting epitopes at the N-terminus (N19) within exon 4 (WFS) and C-terminus (C-20) of BARD1 were used (FIG. 6). For ovarian cancer, it was observed that WSF only weakly reacted with all samples, whereas C20 reacted more strongly with all samples. Overall, the N19 epitope seemed to be less abundant than the C20 epitope but more abundant than the WSF epitope. This indicates that there was little or no FL present but instead that there both splice and omega iso forms were present. Interestingly, the loss of N19 reactivity mostly happened in cancer of T3 stage or cancers with lymph node metastasis (FIG. 8) indicating that omega isoforms correlate with T3 stage and metastatic stages of ovarian cancer. Loss of N-terminus (N-19) is correlated with advanced tumor stage and lymph node metastasis 8D. Furthermore, it was found that both N19 and C20 were highly expressed in clear cell carcinoma, which is the type of ovarian cancer with worst prognosis (FIG. 9A), but not for WFS. This indicates that the over-expression of splice is forms is more prevalent in clear cell carcinoma and correlates with the worst prognosis. Expression of isoforms delta, phi, epsilon, but not FL correlated with clear cell carcinoma. This expression pattern was consistent with the expressed iso form φ and δ. The RT-PCR performed in ovarian cancer cell lines derived from clear cell carcinoma confirmed this hypothesis. Isoforms φ, δ, and ε were highly expressed in SK-OV-3 and TOV-21G cell lines, which are of clear cell type (FIG. 10B).

### Example 6

### BARD1 Isoforms Role in Tumour Cell Growth

It has been previously shown that rat ovarian cancer cells NuTu-19 do not express FL BARD1 but abundantly express the alternatively spliced isoform BARD1 β and δ (Feki et al., 2005, above). NuTu/19 cells are resistant to apoptosis, but exogenous expression of wild-type BARD1 can induce apoptosis in these cells (Feki et al., 2005, above), consistent with the finding that regions of BARD1 that are required for apoptosis is missing in BARD1 isoform δ.

To elucidate the function of BARD1 iso forms, lentiviral vectors containing inducible BARD1 siRNAs, and co-expression of GFP were transduced to NuTu cells to repress BARD1 expression. Si78 which targets the sequence in exon 9 was used to repress BARD1 expression, and si34, which targeted human sequence but not the rat version in exon 2 was used as a control. As shown on Western Blot probed with BARD1 antibody H300 in NuTu cells, NuTu cells do not express FL BARD1, but it expressed isoforms 0 and the smaller bands which correspond to φ and δ (FIG. 10A). After transduction and induction of siRNAs, RT-PCR was performed and si78 completely repressed BARD1 expression (FIG. 10B). Then, fluorescence microscopy showing GFP expression and DAPI staining showed that NuTu cells transduced with si78 showed very few growing cells, and cells became big and flat and stopped proliferating. More importantly, si78-expressing but not si3-expressing cells stopped growing and detached. Cells transduced with si34 looked normal and proliferated (FIGS. 8C and D). SiRNA78 expression lead to growth arrest, siRNA34 had no effect. These experiments demonstrate that BARD1 β and δ are important for NuTu cell growth, and repressing these iso forms leads to a blockage of cell proliferation and subsequently cell death. BARD1 splice isoforms are thus causally involved in cancer-related cell proliferation. Therefore inhibiting these splice iso forms inhibits cell proliferation and leads to cell death. Molecules that reduce BARD1 splice isoform activity should act as cancer therapeutics by stopping cancer cell proliferation and killing these cells.

### Material and Methods

### Cancer Cell Lines

Breast cancer cell lines (B1-B26): MCF-7, MM231, T47D, Hs578T, SKBR3, MM435s, ZR-75-1, BT549, MM453, BT474, PA1, A2780ADR, BT20, HBL100, HMEC, MCF12A, MCF10A, MCF7/6, MCF12F, MM134VI, MM157, MM175VII, MM330, MM468, UCAA812, MM361.

Cervical cancer cell lines (C1-C9): HeLa, SW756, GH354, Ca Ski, C-4 I, C-33 A, HT-3, ME-180, SiHa.

Endometrial cancer cell line (E1-E9): KLE, RL95-2, AN3 CA, HEC-1-B, Ishikawa, Colo. 684, HEC-50, EN, EJ.

Ovarian cancer cell line (O1-O32): A2780, Caov-3, ES-2, NIH: OVCAR-3, SK-OV-3, TOV-21G, TOV-112D, OV-90, OV-MZ-1a, OV-MZ-1c, OV-MZ-2, OV-MZ-2a, OV-MZ-5, OV-MZ-6, OV-MZ-8, OV-MZ-9, OV-MZ-10, OV-MZ-12, OV-MZ-12b, OV-MZ-17b, OV-MZ-18, OV-MZ-20, OV-MZ-21, OV-MZ-22, OV-MZ-26, OV-MZ-27, OV-MZ-30, OV-MZ-32, OV-MZ-33, OV-MZ-35, OV-MZ-37, OV-MZ-38.

### RNA Isolation and RT-PCR

Total RNA from cell lines and tissue specimens were extracted by isopycnic centrifugation as described previously (Kury et al., 1990, Oncogene 5, 1403-1408). For reverse transcription, 0.5 μg of RNA was used in 20 μl of reverse transcription buffer containing 1 μl of random primer, 1.25 μl of 10 mM dNTP's, 1 μl of M-MLV-Powerscript enzyme. The reaction took place at 65° C. 3 minutes followed by 55° C. 60 minutes and 94° C., 5 minutes. cDNA (2-4 μl) was used as a template for PCR with different primers (Table 4 below). It was performed with Taq polymerase in a final volume of 50 μl. Primary denaturation (94° C., 3 min) and final extension (72° C., 10 min) were the same for each PCR Annealing temperature and extension time were variable according to different primers. PCR product (15 μl) was used for analysis in 1% of agarose/TAE gel with EtBr and visualized under UV light.

TABLE 4

| Forward primer | | Reverse primer | | | | |
|---|---|---|---|---|---|---|
| | Position | | Position | PCR | Annealing | |
| | (bp) | | (bp) | product | Temp | Extension |
| Sequence | (exon) | Sequence | (exon) | (bp) | (° C.) | (sec) |
| SEQ ID NO: 31 | −28 | SEQ ID NO: 29 | 1481 | 1508 | 56 | 100 |
| | (exon 1) | | (exon 6) | | | |
| | | SEQ ID NO: 32 | 2333 | 2361 | 56 | 140 |
| | | | (exon 11) | | | |
| SEQ ID NO: 33 | 228 | SEQ ID NO: 32 | 2333 | 2105 | 56 | 130 |
| | (exon 3) | | (exon 11) | | | |
| SEQ ID NO: 34: | 783 | SEQ ID NO: 32 | 2333 | 1550 | 56 | 100 |
| | (exon 4) | | (exon 11) | | | |
| SEQ ID NO: 35 | 985 | SEQ ID NO: 32 | 2333 | 1348 | 57 | 90 |
| | (exon 4) | | (exon 11) | | | |
| SEQ ID NO: 36 | 1280 | SEQ ID NO: 32 | 2333 | 1053 | 54 | 80 |
| | (exon 4) | | (exon 11) | | | |
| SEQ ID NO: 37 | 1378 | SEQ ID NO: 32 | 2333 | 955 | 54 | 70 |
| | (exon 4) | | (exon 11) | | | |
| SEQ ID NO: 38 | 1441 | SEQ ID NO: 32 | 2333 | 892 | 56 | 60 |
| | (exon 6) | | (exon 11) | | | |

Determination of BARD1 cDNA 5′ Ends in Ovarian Cancer

GeneRacer™ Kit (invitrogen) was used to amplify 5′ cDNA end for RNA of ovarian cancer patient and Hela cells. Total RNA (4.5 μg) ovarian cancer and Hela cells were used. Then treated the total RNA with calf intestinal phosphatise (CIP) to dephosphorylate non-mRNA or truncated mRNA. Remove the mRNA 5′ cap structure and ligate the RNA oligo to decapped mRNA. Then reverse transcribing was performed to get the cDNA. In order to amplify the 5′ cDNA end, first PCR was performed with 5′ race primer of SEQ ID NO: 26 (5′-CGACTGGAGCACGAGGACACTGA-3′) and reverse primer in exon 11 of SEQ ID NO: 27 (5′-GTTGCCAAAGCTGTTTG-3). 5′ nested PCR was performed with 5′ nested primer of SEQ ID NO: 28 (5′-GGACACTGACATGGACTGAAGGAGTA-3′) and reverse primer in exon 6 of SEQ ID NO: 29 (5′-TTTTGATACCCGGTGGTGTT-3′). All these procedures were performed according to the manufacturer's instructions. The PCR bands of 5′ nested PCR were loaded on 1% low melting gel, cut, and purified with the QIAEX II kit (Qiagen, Hombrechtikon, Switzerland) followed by sequencing with 5′ nested primer and reverse primer.

### Western Blots

BARD1 antibodies H300 (sc-7372; Santa Cruz, Calif.) was used to detect the N terminus. A synthetic peptide with the sequence GLRPVDYTDDESMKSLLL (SEQ ID NO: 30) within exon 7 of BARD1 was used to generate polyclonal antibodies designated JH3 in rabbits, and was used to detect the C terminus. Protein extracts from different ovarian cancer cells lines were prepared and 40 μg of protein per lane were loaded on 10% SDS-PAGE and blotted onto nylon filters. Membranes were blocked with 5% milk powder in TBS. Antibody incubated with purified anti-Bard1 H300 and JH3 in a 1:500 dilution. Secondary anti-rabbit peroxidase-coupled antibodies were applied in a 1:10,000 dilution. Signal detection was performed with the enhanced chemiluminescence kit (Amersham, Arlington Heights, Ill.).

### Immunohistochemistry

Formalin-fixed and paraffin-embedded micro tissure array were deparaffinized with xylene for 48 hours, and rehydrated through descending alcohol (100% alcohol, 95% alcohol, 70% alcohol, H₂O). The sections were boiled 5 minutes in microwave for antigen retrieval, and then blocking the endogenous peroxidase. Slides were incubated 24 hours at 4° C. in a humidifying chamber with first antibody after BSA (bovine serum albumin) blocking the nonspecific proteins. The primary antibodies used for BARD1 detection were N19 (se-7373, Santa Cruz Biotechnology) WFS described previously (Irminger-Finger et al., 1998, The Journal of cell biology 143, 1329-1339), and C20 (sc-7372, Santa Cruz, Calif.), which recognize N-terminal, epitope in exon 4, and C-terminal epitopes of BARD1, respectively. Secondary antibodies (goat anti-rabbit or rabbit anti-goat) conjugated with horse radish peroxidase (HRP) were applied in 1:100 dilutions at room temperature for 1 hr. Then diaminobenzidine (DAB) staining was permitted for 15 min at room temperature. Slides were counter stained with hematoxylin before dehydration and mounting.

To quantify BARD1 expressing, staining was scored by intensity and percentage of the stained cells. The value of staining intensity and positive cell percentage times together gets the final staining score.

### Clinical Data

Ovarian cancer specimens were obtained from Austria. The pathological diagnosis were made by experienced pathologists and staged according to the WHO and AJCC classification. 106 cases of ovarian cancer from 32-87 year old women, were analyzed, comprising of 60 cases of serous carcinoma, 24 cases of endomeriod carcinoma, 16 cases of mucinous carcinoma, and 6 cases of clear cell carcinoma. According to TNM staging system, there were 38 cases in T1; 15 cases in T2; 53 cases in T3; 39 cases in N0, and 67 cases in N1 stage. There were 25, 26, and 55 cases of pathologic grade 1 to 3, respectively.

### BARD1 Repression in NuTu Cells

NuTu cell culture—as described in literature

siRNA—standard methods

Transfection of NuTu cells—standard methods

Fluorescence microscopy—standard methods.

1-57. (canceled)

58. A method for detecting the presence of gynaecological cancer related proteins in a biological sample, comprising the steps of:
a) Determining one or more of the following in a sample from a female mammal:
(i) The expression level of a protein of SEQ ID NO: 1 through a detectable signal proportional to the said level of expression;
(iia) The expression level of at least one protein comprising an amino acid sequence selected from the group consisting of SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9 through a detectable signal proportional to the said level of expression; and/or
(iib) The expression and/or expression level of at least one protein comprising an amino acid sequence selected from the group consisting of SEQ ID NO: 10; SEQ ID NO: 11 and SEQ ID NO: 24 through a detectable signal proportional to the said level of expression; and
b) Detecting a signal indicative of a ratio lower than a 1:1 ratio between the expression level obtained under step (i) and the expression level obtained under steps (iia) and/or (iib); or detecting a signal indicative of the expression/expression level determined under step (iib).

59. The method according to claim 58, comprising the steps of:
a) Determining one or more of the following in a sample from a female mammal:
(i) The expression level of a protein of SEQ ID NO: 1 through a detectable signal proportional to the said level of expression; and/or
(iia) The expression level of at least one protein of an amino acid sequence selected from the group consisting of SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9 through a detectable signal proportional to the said level of expression; and
b) Detecting a signal indicative of a ratio lower than a 1:1 ratio between the expression level obtained under step (i) and the expression level obtained under step (iia).

60. The method according to claim 58 comprising the steps of:
a) Determining one or more of the following in a sample from a female mammal:
(i) The expression level of a protein of SEQ ID NO: 1 through a detectable signal proportional to the said level of expression; and
(iib) The expression and/or expression level of at least one protein of an amino acid sequence selected from the group consisting of SEQ ID NO: 10; SEQ ID NO: 11 and SEQ ID NO: 24 through a detectable signal proportional to the said level of expression; and
b) Detecting a signal indicative of the expression/expression level obtained under step (iib).

61. The method according to claim 60, wherein the signal obtained under the detection step (c) is indicative of a gynaecological cancer or a risk of developing a future gynaecological cancer in the subject.

62. The method according to claim 58, further comprising comparing the expression levels obtained under step (i) with the expression level obtained under steps (iia) and/or (iib).

63. The method according to claim 59, further comprising comparing the expression levels obtained under step (i) with the expression level obtained under steps (iia) and/or (iib).

64. The method according to claim 58, wherein the biological sample is blood.

65. A method for detecting the presence of gynaecological cancer related proteins in a biological sample, comprising the steps of:
a) Reacting a sample from a female mammal with at least one antibody, a fragment thereof or a combination thereof, which is specific to a protein of SEQ ID NO: 1;
b) Reacting the said sample with at least one antibody, fragment thereof, or a combination thereof, which is specific to at least one protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9;
c) Reacting the said sample with at least one antibody, a fragment thereof or a combination thereof, which is specific to at least one protein comprising an amino acid sequence selected from SEQ ID NO: 10, SEQ ID NO: 11 and SEQ ID NO: 24;
d) Detecting (a) a protein of SEQ ID NO: 1; and (b) a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9; and/or (c) a protein comprising an amino acid sequence selected from SEQ ID NO: 10, SEQ ID NO: 11 and SEQ ID NO: 24; wherein the detection is achieved through the detection of the interaction of each said antibody, fragment thereof or combination thereof, used under steps (i) and (ii) and/or (iii) with the corresponding said at least one protein, wherein the presence of the interaction correlates with the concentration of the protein in the biological sample; and
e) Detecting a signal indicative of a ratio lower than a 1:1 ratio between the said interaction detection signal obtained under step (iv) for a protein of SEQ ID NO: 1 and the said interaction detection signal obtained under step (iv) for either a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9; or for a protein comprising an amino acid sequence selected from SEQ ID NO: 10, SEQ ID NO: 11 and SEQ ID NO: 24; or detecting a signal indicative of an interaction signal detected under step (iv) for a protein comprising an amino acid sequence selected from SEQ ID NO: 10, SEQ ID NO: 11 and SEQ ID NO: 24.

66. The method according to claim 65, wherein the signal detected under step (v) indicative of a ratio lower than a 1:1 ratio between the said interaction detection signal obtained under step (iv) for a protein of SEQ ID NO: 1 and the said interaction detection signal obtained under step (iv) for a protein comprising an amino acid sequence selected from SEQ ID NO: 2, SEQ ID NO: 3, SEQ ID NO: 4, SEQ ID NO: 5, SEQ ID NO: 6, SEQ ID NO: 7; SEQ ID NO: 8 and SEQ ID NO: 9, is indicative of a gynaecological cancer.

67. The method according to claim 65, wherein the signal detected under step (v) indicative for a protein comprising an amino acid sequence selected from SEQ ID NO: 10, SEQ ID NO: 11 and SEQ ID NO: 24, is indicative of a gynaecological cancer or a risk of developing a future gynaecological cancer in the subject.

68. The method according to claim 65, wherein the antibodies used under step (ii) is a combination of antibodies wherein the combination comprises (a) at least one antibody against exon 1 from full length BARD1 (SEQ ID NO: 12); (b) at least one antibody against one exon selected from exon 4, exon 5, exon 6, exon 7, exon 8 and exon 9 from full length BARD1 (SEQ ID NO: 12); and (c) at least one antibody against exon 10 and/or exon 11 from full length BARD 1 (SEQ ID NO: 12).

69. An isolated polypeptide comprising at least one sequence of amino acids having at least 80% identity or homology with a sequence comprising SEQ ID NO: 2, SEQ ID NO: 3, SEQ ID NO: 4, SEQ ID NO: 5, SEQ ID NO: 6, SEQ ID NO: 7; SEQ ID NO: 8; SEQ ID NO: 9; SEQ ID NO: 10, SEQ ID NO: 11 and SEQ ID NO: 24.

70. An isolated nucleic acid consisting of a nucleotide sequence encoding a polypeptide according to claim 69.

71. An isolated nucleic acid according to claim 70, said nucleic acid selected from the group consisting of SEQ ID NO: 13; SEQ ID NO: 14; SEQ ID NO: 15; SEQ ID NO: 16; SEQ ID NO: 17; SEQ ID NO: 18, SEQ ID NO: 19; SEQ ID NO: 20 and SEQ ID NO: 23.

72. A method of expressing a polypeptide comprising culturing a recombinant host cell expressing a nucleic acid according to claim 70 under conditions that allow for the expression of a polypeptide.

73. A probe or primer that hybridizes with a nucleic acid according to claim 70.

74. An isolated antibody that selectively binds at least one polypeptide according to claim 69.

75. A combination of antibodies comprising (a) at least one antibody against exon 1 from full length BARD 1 (SEQ ID NO: 12); (b) at least one antibody against one exon selected from exon 4, exon 5, exon 6, exon 7, exon 8 and exon 9 from full length BARD 1 (SEQ ID NO: 12); and (c) at least one antibody against exon 10 and/or exon 11 from full length BARD 1 (SEQ ID NO: 12).

76. A method for detecting the level of cellular expression of proteins of comprising the step of:
(i) Contacting at least one antibody according to claim 74 or a fragment thereof, or a combination of antibodies according to claim 75 with cells to be tested under appropriate conditions for binding of the said antibodies, combination thereof or combination of antibodies to at least a protein comprising SEQ ID NO: 2, SEQ ID NO: 3, SEQ ID NO: 4, SEQ ID NO: 5, SEQ ID NO: 6, SEQ ID NO: 7; SEQ ID NO: 8; SEQ ID NO: 9; SEQ ID NO: 10, SEQ ID NO: 11 and SEQ ID NO: 24 on the cells;
(ii) Determining the level of binding of said at least one antibody, combination thereof, or combination of antibodies to the cell as indicative level of expression of the said protein.

77. A recombinant expression vector comprising a nucleic acid molecule according to claim 70, wherein the vector optionally comprises an expression control sequence, allowing expression in prokaryotic or eukaryotic host cells of the encoded polypeptide, operably linked to the nucleic acid molecule.

78. An immunoassay kit for detecting gynaecological cancer in a biological sample, the kit comprising at least one antibody according to claim 74 or a fragment thereof or a combination of antibodies according to claim 75.

79. A method of treating a disease comprising the administration of a therapeutically effective amount of an antagonist of a polypeptide according to claim 69 in a mammal in need thereof; wherein the disease is a gynaecological cancer, including breast, ovarian, cervical and uterine cancers.

80. The method according to claim 79, wherein the antagonist is a SiRNA and whereby tumor growth is arrested.


SEQUENCE LISTING (38 sequences)

SEQ ID NO: 1 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 2 - protein, 758 aa, Homo sapiens [sequence omitted]

SEQ ID NO: 3 - protein, 680 aa, Homo sapiens [sequence omitted]

SEQ ID NO: 4 - protein, 732 aa, Homo sapiens [sequence omitted]

SEQ ID NO: 5 - protein, 127 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSGRHTFC

SEQ ID NO: 6 - protein, 326 aa, Homo sapiens [sequence omitted]

SEQ ID NO: 7 - protein, 263 aa, Homo sapiens
MPDNRQPRNRQPRIRSGNEPRSASAMEPDGRGAWAHSRAALDRLEKLLRCSRCTNILREPVCLGGCEHIFCSNCVSDCIGTGCPVCYTPAWIQDLKINRQLDSMIQLCSKRNLLHDNELSGVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 8 - protein, 167 aa, Homo sapiens [sequence omitted]

SEQ ID NO: 9 - protein, 219 aa, Homo sapiens
ASLWFPEASLLPALRGAFHPKAGRCRIIGSRGTGSRGSAPGTSLVPRPPWNRMVAVPGPTVAPRSTAWRSCCAARVGVKACLRRKVCEQEEKYEIPEGPRRSRLNREQLLPKLFDGCYFYLWGTFEHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 10 - protein, 624 aa, Homo sapiens
VRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 11 - protein, 449 aa, Homo sapiens
SSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 12 - DNA, 2530 bp, Homo sapiens
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 13 - DNA, 2473 bp, Artificial
  /note=“synthetic construct”
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 14 - DNA, 2324 bp, Artificial
  /note=“synthetic construct”
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 15 - DNA, 1456 bp, Artificial
  /note=“synthetic construct”
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagcgtgttacaccccggcctggatacaagacttgaagataaatagacaactggacagcatgattcaactttgtagtaagcttcgaaatttgctacatgacaatgagctgtcagggcgacataccttctgttgaataccttttacaaaatggaagtgatccaaatgttaaagaccatgctggatggacaccattgcatgaagcttgcaatcatgggcacctggaggtagtggaattattgctccagcataaggcattggtgaacaccaccgggtatcaaaatgactcaccacttcacgatgcagccaagaatgggcacatggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctg

SEQ ID NO: 16 - DNA, 1010 bp, Artificial
  /note=“synthetic construct”
gaggagcctttcatccgaaggcgggacgatgccggataatcggcagccgaggaaccggcagccgaggatccgctccgggaacgagcctcgttccgcgtccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaa

SEQ ID NO: 17 - DNA, 780 bp, Homo sapiens
gaggagcctttcatccgaaggcgggacgatgccggataatcggcagccgaggaaccggcagccgaggatccgctccgggaacgagcctcgttccgcgtccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcaggggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttat

SEQ ID NO: 18 - DNA, 702 bp, Artificial
  /note=“synthetic construct”
agacgcgggaagagcttggccggtttcgagtcgctgacctgcagcttccctgtggttccccgaggcctccttgcttcccgctctccgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttggggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcgaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctg

SEQ ID NO: 19 - DNA, 2000 bp, Homo sapiens
aagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 20 - DNA, 1475 bp, Homo sapiens
tttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 21 - protein, 24 aa, Artificial
  /note=“synthetic construct”
MVAVPGPTVAPRSTAWRSCCAARV

SEQ ID NO: 22 - protein, 6 aa, Artificial
  /note=“synthetic construct”
GRHTFC

SEQ ID NO: 23 - DNA, 1168 bp, Homo sapiens
tttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 24 - protein, 347 aa, Homo sapiens
LLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 25 - protein, 30 aa, Artificial
  /note=“synthetic construct”
LKEDKPRKSLFNDAGNKKNSIKMWFSPRSK

SEQ ID NO: 26 - DNA, 23 bp, Artificial
  /=“synthetic construct”
cgactggagcacgaggacactga

SEQ ID NO: 27 - DNA, 17 bp, Artificial
  /note=“synthetic construct”
gttgccaaagctgtttg

SEQ ID NO: 28 - DNA, 26 bp, Artificial
  /note=“synthetic construct”
ggacactgacatggactgaaggagta

SEQ ID NO: 29 - DNA, 20 bp, Artificial
  /=“synthetic construct”
ttttgatacccggtggtgtt

SEQ ID NO: 30 - protein, 18 aa, Artificial
  /note=“synthetic construct”
GLRPVDYTDDESMKSLLL

SEQ ID NO: 31 - DNA, 20 bp, Artificial
  /note=“synthetic construct”
gaggagcctttcatccgaag

SEQ ID NO: 32 - DNA, 21 bp, Artificial
  /note=“synthetic construct”
cagctgtcaagaggaagcaac

SEQ ID NO: 33 - DNA, 20 bp, Artificial
  /note=“synthetic construct”
gtgactgcattggaactgga

SEQ ID NO: 34 - DNA, 20 bp, Artificial
  /note=“synthetic construct”
agcaagtggctccttgacag

SEQ ID NO: 35 - DNA, 24 bp, Artificial
  /note=“synthetic construct”
ccagtcccatttctaagagatgta

SEQ ID NO: 36 - DNA, 18 bp, Artificial
  /note=“synthetic construct”
gaggagagactttgctcc

SEQ ID NO: 37 - DNA, 18 bp, Artificial
  /note=“synthetic construct”
gctggatggacaccattg

SEQ ID NO: 38 - DNA, 20 bp, Artificial
  /note=“synthetic construct”
ctccagcataaggcattggt